(19) United States
(12) Patent Application Publication
(10) Pub. No.: US 2012/0302636 A1
(43) Pub. Date: Nov. 29, 2012

(54) METHOD OF TREATING A DISORDER ASSOCIATED WITH MTP

(76) Inventors: Harutoshi KON, Tokyo (JP); Yoshiro Masuda, Tokyo (JP); Hiromitsu Watanabe, Tokyo (JP)

(21) Appl. No.: 13/328,787

(22) Filed: Dec. 16, 2011

Related U.S. Application Data
(63) Continuation of application No. PCT/US10/38965, filed on Jun. 17, 2010.
(60) Provisional application No. 61/268,925, filed on Jun. 18, 2009.

(51) Int. Cl. A61K 31/216; A61P 9/00; A61P 9/12; A61P 3/04; A61P 3/10; A61P 3/06; A61P 3/00

(52) U.S. Cl. 514/533

(57) ABSTRACT

Provided is a method of treating or preventing a disorder in a subject comprising administering twice per day a therapeutically effective amount of a compound of formula (I)

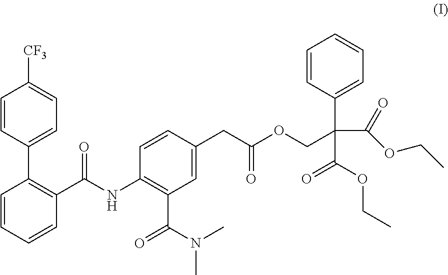

or a pharmaceutically acceptable salt, solvate, or hydrate thereof, to a subject that has or is at risk of having a disorder selected from the group consisting of hyperlipidemia, arteriosclerosis, coronary artery diseases, metabolic syndrome, obesity, diabetes, prediabetes, and hypertension, whereupon the disorder is treated or prevented in the subject. The method optionally comprises administering a therapeutically effective amount of the compound of formula (I), or a pharmaceutically acceptable salt, solvate, or hydrate thereof, to the subject, in which the therapeutically effective amount is increased once or twice during the course of administration.

### BACKGROUND OF THE INVENTION

Microsomal triglyceride transfer protein (MTP) exists in the microsome fractions of hepatocytes and intestinal epithelial cells, and catalyzes the transfer of triglyceride (TG) or cholesteryl ester in cells. Accordingly, the control of lipoproteins, such as TG, cholesterol, and low-density lipoprotein (LDL), in blood and/or the control of lipids in cells by adjusting the activity of MTP is believed to provide a treatment or prophylaxis of hyperlipidemia, arteriosclerosis, coronary artery diseases, metabolic syndrome, obesity, diabetes, prediabetes, and hypertension. However, with the development of MTP inhibitors, some cases of fatty liver were reported, and concern over hepatotoxicity has been raised. For these reasons, there is a need for methods of treating and/or preventing disorders associated with MTP that are efficacious with a reduction or removal of the side effects associated with known treatments.

### BRIEF SUMMARY OF THE INVENTION

The invention provides a method of treating or preventing a disorder in a subject comprising administering twice per day a therapeutically effective amount of a compound of formula (I)

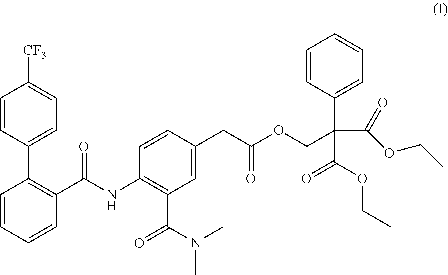

or a pharmaceutically acceptable salt, solvate, or hydrate thereof, to a subject that has or is at risk of having a disorder selected from the group consisting of hyperlipidemia, arteriosclerosis, coronary artery diseases, metabolic syndrome, obesity, diabetes, prediabetes, and hypertension, whereupon the disorder is treated or prevented in the subject.

The method optionally comprises administering twice per day a therapeutically effective amount of the compound of formula (I), or a pharmaceutically acceptable salt, solvate, or hydrate thereof, to the subject, in which the therapeutically effective amount is increased once or twice during the course of the administration of the compound of formula (I), or a pharmaceutically acceptable salt, solvate, or hydrate thereof. In one embodiment, the method comprises (a) administering twice per day a first therapeutically effective amount of the compound of formula (I), or a pharmaceutically acceptable salt, solvate, or hydrate thereof, to the subject for a first time period, and (b) administering twice per day a second therapeutically effective amount of the compound of formula (I), or a pharmaceutically acceptable salt, solvate, or hydrate thereof, to the subject for a second time period after the first time period, wherein the second therapeutically effective amount is greater than the first therapeutically effective amount. In another embodiment, the method comprises (a) administering twice per day a first therapeutically effective amount of the compound of formula (I), or a pharmaceutically acceptable salt, solvate, or hydrate thereof, to the subject for a first time period, (b) administering twice per day a second therapeutically effective amount of the compound of formula (I), or a pharmaceutically acceptable salt, solvate, or hydrate thereof, to the subject for a second time period after the first time period, and (c) administering twice per day a third therapeutically effective amount of the compound of formula (I), or a pharmaceutically acceptable salt, solvate, or hydrate thereof, to the subject for a third time period after the second time period, wherein the second therapeutically effective amount is greater than the first therapeutically effective amount, and the third therapeutically effective amount is greater than the second therapeutically effective amount.

In certain embodiments, the invention provides that the compound is administered to the subject with food or when the subject is in a fed state.

### DETAILED DESCRIPTION OF THE INVENTION

The compound of formula (I), or a pharmaceutically acceptable salt, solvate, or hydrate thereof, is an MTP inhibitor that does not cause undesirable side effects often associated with certain MTP inhibitors, such as fatty liver.

The compound of formula (I) is chemically known as diethyl 2-((2-(3-(dimethylcarbamoyl)-4-(4′-(trifiuoromethyl)biphenyl-2-ylcarboxamido)phenyl)acetoxy) methyl)-2-phenyl malonate. The compound of formula (I) can be prepared by any suitable synthesis, including the synthesis described in U.S. Patent Application Publication 2005/0075367, the entirety of which is incorporated herein by reference. As disclosed in Burnett (IDrugs, 9(7): 495-499 (2006)), the compound of formula (I) may be prepared, for example, according to the following procedure. 5-Chloro-2-nitrobenzoic acid is converted to its dimethylamide derivative via the acid chloride. The amide is treated with t-butyl methyl malonate to provide a 2-(4-nitrophenyl) malonate derivative. Selective hydrolysis and decarboxylation of the derivative yields the nitrophenyl acetic acid methyl ester, which can be reduced to its corresponding aniline. A reaction of the aniline with the acid chloride of 4′-trifluoromethyl-2-biphenylcarboxylic acid, and a hydrolysis of the ester, provide the biphenyl-2-carbonylaminophenyl acetic acid. Coupling of the acid with diethyl 2-(hydroxymethyl)-2-phenyl malonate in the presence of 4-dimethylaminopyridine and 1-ethyl-3-(3′-diethylaminopropyl)carbodiimide in dichloromethane at room temperature for 6 h produces the compound of formula (I).

The compound of formula (I), or a pharmaceutically acceptable salt, solvate, or hydrate thereof, is administered in a pharmaceutical composition comprising the compound and a pharmaceutically acceptable carrier. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

If desired, pharmaceutical additives such as a lubricant, a disintegrating agent, a solvent, a solubilizer, a suspending agent, a suspending agent, an isotonizing agent, a buffer, a soothing agent, a preservative, an antioxidant, a sweetening agent, or a coloring agent can also be used. Suitable examples of the excipient include lactose, sucrose, D-mannitol, starch, crystalline cellulose, and light anhydrous silicic acid. Suitable examples of the lubricant include magnesium stearate, calcium stearate, talc, and colloidal silica. Suitable examples of the binder include crystalline cellulose, sucrose, D-mannitol, dextrin, hydroxypropyl cellulose, hydroxypropyl methylcellulose, and polyvinylpyrrolidone. Suitable examples of the disintegrating agent include starch, carboxymethylcellulose, carboxymethylcellulose calcium, crosscarmellose sodium, and sodium carboxymethylstarch. Suitable examples of the solvent include water for injection, alcohol, propylene glycol, macrogol, sesame-seed oil, corn oil, and propylene glycol fatty acid ester. Suitable examples of the solubilizer include polyethyleneglycol, propyleneglycol, D-mannitol, benzyl benzoate, ethanol, trisaminomethane, cholesterol, triethanolamine, sodium carbonate, and sodium citrate. Suitable examples of the suspending agent include surfactants (e.g., stearyl triethanolamine, sodium lauryl sulfate, lauryl aminopropionic acid, lecithin, benzalkonium chloride, benzethonium chloride, and glycerin monostearate), polyvinyl alcohol, polyvinylpyrrolidone, carboxymethylcellulose sodium, methylcellulose, and hydroxymethyl cellulose. Suitable examples of the isotonizing agent include sodium chloride, glycerin, and D-mannitol. Suitable examples of the buffer include phosphate, acetate, carbonate, and citrate. Suitable examples of the soothing agent include benzyl alcohol. Suitable examples of the preservative include paraoxybenzoic acid esters, chlorobutanol, benzyl alcohol, phenethyl alcohol, dehydroacetic acid, and sorbic acid. Suitable examples of the antioxidant include sulfites and ascorbic acid. Suitable examples of the sweetening agent include aspartame, saccharin sodium, and stevia. Suitable examples of the coloring agent include food colors such as food yellow No. 5, food red No. 2, and food blue No. 2, lake colors for food, and iron oxide.

The pharmaceutical composition comprising the compound of formula (I) can be provided in any suitable formulation, such as tablets, capsules, pills, powders, granules, suppositories, injections, eye drops, solutions, capsules, troches, aerosols, elixirs, suspensions, emulsions, and syrups. In certain embodiments, the pharmaceutical composition is administered as an oral formulation, such as a tablet or capsule. In a preferred embodiment, the oral formulation is a tablet (e.g., a tablet prepared in accordance with the methods and compositions described in U.S. Patent Application Publication 2006/0153913A1, the entire contents of which are incorporated herein by reference).

When the compound of formula (I), or a pharmaceutically acceptable salt, solvate, or hydrate thereof, is formulated into a solid preparation, such as a tablet, pill, powder, or granules, additives such as lactose, mannitol, glucose, hydroxypropyl cellulose, microcrystalline cellulose, starch, polyvinylpyrrolidone, magnesium aluminometasilicate, or powdery silicic anhydride can be used. When the compound of formula (I), or a pharmaceutically acceptable salt, solvate, or hydrate thereof, is formulated into a tablet or pill, it can be coated with a gastroenteric or enteric coating film containing a substance such as sucrose, gelatin, hydroxypropyl cellulose, or hydroxymethyl cellulose phthalate. Furthermore, the tablet or pill can be a multi-layered tablet or pill comprising two or more layers.

Suitable oral formulations include capsules that comprise liquid, semi-solid, or solid contents. Liquid or semi-solid contents can be prepared by dissolving the compound of formula (I), or a pharmaceutically acceptable salt, solvate, or hydrate thereof, in a solvent and adding an additive thereto. Examples of solvents are purified water, ethanol, and vegetable oil. Ethanol or a mixture of purified water and ethanol preferably is used. Suitable additives for a capsule include, for example, propylene glycol fatty acid esters; low molecular weight polyethylene glycols such as polyethylene glycol 200 to 600 g/mol, glycerine fatty acid esters thereof, and medium chain fatty acid triglycerides thereof; alcohols/polyols such as stearyl alcohol, cetanol, polyethylene glycol, or esters thereof; lipids such as sesame oil, soy bean oil, peanut oil, corn oil, hydrogenated oil, paraffin oil, bleached wax; and fatty acids such as triethyl citrate, triacetin, stearic acid, palmitic acid, myristic acid. These additives are suitable for preparing liquid or semi-solid contents of the capsule.

Propylene glycol fatty acid esters are a preferable additive in capsule formulations. Examples of the propylene glycol fatty acid esters are propylene glycol monocaprylate (CAPMUL™ PG-8, SEFOL™ 218, and CAPRYO™ 190), propylene glycol monolaurate (LAUROGLYCOL™ FCC), propylene glycol monooleate (MYVEROL™ P-06), propylene glycol myristate, propylene glycol monostearate, propylene glycol lisinolate (PROPYMULS™), propylene glycol dicaprylate/dicaprate (CAPTEX™ 200) propylene glycol dilaurate, propylene glycol distearate, and propylene glycol dioctanoate (CAPTEX™ 800). Although there is no particular limitation to the materials constituting the capsules of the invention, the materials can include, for example, polysaccharides derived from natural products such as agar, alginic acid salt, starch, xanthan, and dextran; proteins such as gelatin and casein; chemically processed products such as hydroxystarch, pullulan, hydroxypropyl cellulose, hydroxypropyl methylcellulose, polyvinyl alcohol or derivatives thereof, polyacryl derivatives, polyvinylpyrrolidone or derivatives thereof, and polyethylene glycol.

When the pharmaceutical composition is a liquid formulation for oral administration, such as a pharmaceutically acceptable emulsion, solubilizer, suspension, syrup, or elixir, suitable diluents include, for example, purified water, ethanol, vegetable oils, and emulsifiers. In addition, auxiliary agents such as wetting agents, suspending agents, sweeteners, condiments, flavors, or antiseptics can be added to a liquid formulation.

Suitable parenteral formulations, such as injections, can comprise sterilized aqueous or non-aqueous solutions, solubilizers, suspending agents, and emulsifiers. Examples of the aqueous solutions, solubilizers, and suspending agents include distilled water for injections, physiological saline, cyclodextrin, and derivatives thereof; organic amines such as triethanolamine, diethanolamine, monoethanolamine, and triethylamine; and inorganic alkaline solutions. When aqueous solutions are used, substances such as propylene glycol, polyethylene glycol, vegetable oil (e.g., olive oil), or an alcohol (e.g., ethanol) can be further added. Surfactants, such as polyoxyethylene hydrogenated castor oils, sucrose fatty acid esters, or lecithin or hydrogenated lecithin (for liposome formation), can be used as solubilizers. A parenteral formulation can be formulated into an emulsion comprising a non-aqueous solubilizer such as vegetable oil, together with lecithin, polyoxyethylene hydrogenated castor oil, or polyoxyethylene-polyoxypropylene glycol.

A compound of formula (I), or a pharmaceutically acceptable salt, solvate, or hydrate thereof, can be administered to a subject in combination with additional active agents. The additional active agents can be included in the same or different pharmaceutical composition comprising the compound of formula (I). Suitable formulations for the additional active agents are described herein. There is no particular limitation as to the timing for the administration of the additional active agents. For example, the additional active agents can be administered simultaneously or intermittently (e.g., before and/or after) with the compound of formula (I), or a pharmaceutically acceptable salt, solvate, or hydrate thereof.

As additional active agents to be used in combination with the compound of formula (I), drugs for the treatment or prophylaxis of hyperlipidemia, arteriosclerosis, coronary artery diseases, metabolic syndrome, obesity, diabetes, prediabetes, and hypertension, and combinations thereof can be used. Examples of antihyperlipidemic drugs include statin-type drugs, more specifically, lovastatin, simvastatin, pravastatin, fluvastatin, atorvastatin, and cerivastatin. Examples of anti-obesity drugs include mazindol and olristat. Examples of anti-diabetic drugs include insulin preparations, sulfonylurea drugs, insulin secretion-promoter drugs, sulfonamide drugs, biguanide drugs, α-glucosidase inhibitors, and insulin resistance-improving drugs (e.g., insulin, glibenclamid, tolbutamide, glyclopyramide, acetohexamide, glimepiride, tolazamide, gliclazide, nateglinide, glibuzol, metformin hydrochloride, buformin hydrochloride, voglibose, acarbose, and pioglitazone hydrochloride). Examples of the anti-hypertension drugs include loop diuretics, angiotensin converting enzyme inhibitors, angiotensin II receptor antagonists, calcium antagonists, β-blockers, 60 ,β-blockers, and α-blockers (e.g., furosemide delayed release, captopril, captopril delayed release, enalapril maleate, alacepril, delapril hydrochloride, silazapril, lisinopril, benazepril hydrochloride, imidapril hydrochloride, temocapril hydrochloride, quinapril hydrochloride, trandolapril, perindopril erbumine, losartan potassium, candesartan cilexetil, nicardipine hydrochloride, nicardipine hydrochloride delayed release, nilvadipine, nifedipine, nifedipine delayed release, benidipine hydrochloride, diltiazem hydrochloride, diltiazem hydrochloride delayed release, nisoldipine, nitrendipine, manidipine hydrochloride, barnidipine hydrochloride, efonidipine hydrochloride, amlodipine besylate, felodipine, cilnidipine, aranidipine, propranolol hydrochloride, propranolol hydrochloride delayed release, pindolol, pindolol delayed release, indenolol hydrochloride, carteolol hydrochloride, carteolol hydrochloride delayed release, bunitrolol hydrochloride, bunitrolol hydrochloride delayed release, atenolol, asebutolol hydrochloride, metoprolol tartrate, metoprolol tartrate delayed release, nipradilol, penbutolol sulfate, tilisolol hydrochloride, carvedilol, bisoprolol fumarate, betaxolol hydrochloride, celiprolol hydrochloride, bopindolol malonate, bevantolol hydrochloride, labetalol hydrochloride, arotinolol hydrochloride, amosulalol hydrochloride, prazosin hydrochloride, terazosin hydrochloride, doxazosin mesylate, bunazocin hydrochloride, bunazocin hydrochloride delayed release, urapidil, and phentolamine mesylate).

The dosage of additional active agents for combination use can be determined based on their clinical doses, and can be chosen depending on the age, weight, and/or condition of the subject, medication time, dosage form, method of administration, or any combination of the foregoing factors.

The phrase “therapeutically effective amount” means an amount of the compound of formula (I), or a pharmaceutically acceptable salt, solvate, or hydrate thereof, that (i) treats or prevents the particular disorder, (ii) attenuates, ameliorates, or eliminates one or more symptoms of the particular disorder, and/or (iii) prevents or delays the onset of one or more symptoms of the particular disorder described herein. In particular, the therapeutically effective amount of the compound of formula (I), or a pharmaceutically acceptable salt, solvate, or hydrate thereof, is about 0.5 mg to about 5 mg (e.g., about 0.5, 1, 1.5, 2, 2.5, 3, 3.5 4, 4.5, or 5 mg) per dose, which is administered twice a day. The term “about” in the context of dosages means ±0.25 mg of any given amount or range. In certain embodiments, the therapeutically effective amount of the compound of formula (I), or a pharmaceutically acceptable salt, solvate, or hydrate thereof, is about 1 mg to about 5 mg (e.g., about 1, 1.25, 1.5, 1.75, 2, 2.25, 2.5, 2.75, 3, 3.25, 3.5, 3.75, 4, 4.25, 4.5, 4.75, or 5 mg). Preferably, the therapeutically effective amount of the compound of formula (I), or a pharmaceutically acceptable salt, solvate, or hydrate thereof, is about 0.5 mg, about 1 mg, about 1.5 mg, about 2 mg, about 2.5 mg, about 3 mg, about 3.5 mg, about 4 mg, about 4.5 mg, or about 5 mg administered twice per day.

In an embodiment of the inventive method, the compound of formula (I), or a pharmaceutically acceptable salt, solvate, or hydrate thereof, is administered to the subject with food or when the subject is in a fed state. The term “fed state” means the subject has commenced the consumption of food within about 180 minutes prior to administering the compound of formula (I), or a pharmaceutically acceptable salt, solvate, or hydrate thereof. In some instances, the compound is administered to the subject with food. In other instances, the compound is administered to the subject after the subject has begun to consume food (e.g., about 1-180 minutes after the subject has begun to eat). The term “about” in the context of time relative to the consumption of food means ±5 minutes of any given time or range of times. The compound of formula (I), or a pharmaceutically acceptable salt, solvate, or hydrate thereof, can be administered to the subject about 5 minutes, about 10 minutes, about 20 minutes, or about 30 minutes after the subject has begun to consume food. Alternatively, or in addition, the compound of formula (1), or a pharmaceutically acceptable salt, solvate, or hydrate thereof, can be administered to the subject no more than about 180 minutes, no more than about 120 minutes, no more than about 60 minutes, or no more than about 30 minutes after the subject has begun to consume food. Thus, the compound of formula (I), or a pharmaceutically acceptable salt, solvate, or hydrate thereof, can be administered in any time period bounded by any two of the foregoing values, e.g., about 5-180 minutes, about 20-180 minutes, about 30-180 minutes, about 10-120 minutes, about 20-120 minutes, about 20-60 minutes, or about 30-60 minutes. In an especially preferred embodiment, the compound of formula (I), or a pharmaceutically acceptable salt, solvate, or hydrate thereof, is administered about 30 minutes after the subject has begun to consume food. Since the compound of formula (I), or a pharmaceutically acceptable salt, solvate, or hydrate thereof, is administered twice per day, in a preferred embodiment, the compound is administered either with or subsequent to consuming food in time periods that are no less than about four hours (e.g., more than about 4 h, more than about 5 h, more than about 6 h, more than about 7 h, more than about 8 h, more than about 9 h, more than about 10 h) apart. The food desirably is solid food, especially solid food of at least 30 g (e.g., at least 40 g, at least 50 g, at least 75 g, at least 100 g, at least 125 g, or at least 150 g). Preferably the food is part of a meal (e.g., breakfast, lunch, or supper). For example, the compound of formula (I), or a pharmaceutically acceptable salt, solvate, or hydrate thereof, can be administered to the subject either with or after consuming breakfast and supper, in which breakfast and supper are at least about four hours apart.

In an embodiment of the invention, the method contemplates an escalating dosing schedule. More specifically, the method comprises administering twice per day a therapeutically effective amount of the compound of formula (I), or a pharmaceutically acceptable salt, solvate, or hydrate thereof, to the subject, and the therapeutically effective amount is increased once or twice during the course of the administration of the compound of formula (I), or a pharmaceutically acceptable salt, solvate, or hydrate thereof In particular, the method comprises (a) administering a first therapeutically effective amount of the compound of formula (I), or a pharmaceutically acceptable salt, solvate, or hydrate thereof, to the subject for a first time period, and (b) administering a second therapeutically effective amount of the compound of formula (I), or a pharmaceutically acceptable salt, solvate, or hydrate thereof, to the subject for a second time period after the first time period. The method can optionally comprise step (c) administering a third therapeutically effective amount of the compound of formula (I), or a pharmaceutically acceptable salt, solvate, or hydrate thereof, to the subject for a third time period after the second time period. The second therapeutically effective amount is greater than the first therapeutically effective amount, and if used, the third therapeutically effective amount is greater than the second therapeutically effective amount. Typically, the first and second time periods are the same or different and each independently is about 1 to 5 weeks (i.e., about 1 week, about 2 weeks, about 3 weeks, about 4 weeks, or about 5 weeks). Preferably the first and/or second time period independently is about 2 to 4 weeks (e.g., about 2 weeks, about 3 weeks, or about 4 weeks). More preferably, the first and/or second time period is about 2 weeks. The term “about” in this context of time means ±2 days of any given time or range of times.

In a method with an escalating dosing schedule, typically, the first therapeutically effective amount is about 0.5 mg, about 1 mg, about 1.5 mg, about 2 mg, about 2.5 mg, about 3 mg, about 3.5 mg, about 4 mg, about 4.5 mg, or about 5 mg. The second therapeutically effective amount typically is at least 25% greater, at least 50% greater, at least 75% greater, or at least 100% greater than the first therapeutically effective amount. If a third dose is contemplated, the third therapeutically effective amount is at least 25% greater, at least 50% greater, at least 75% greater, or at least 100% greater than the second therapeutically effective amount.

The invention provides a therapeutic agent for the treatment or prophylaxis of a disorder associated with MTP. Examples of disorders associated with MTP include hyperlipidemia, arteriosclerosis, coronary artery diseases, metabolic syndrome, obesity, diabetes, prediabetes, and hypertension. Preferably, the disorder is obesity or diabetes (e.g., type 2 diabetes). Preferably, one or more symptoms of the disorder are prevented, reduced, or eliminated subsequent to administration of the compound of formula (I), or a pharmaceutically acceptable salt, solvate, or hydrate thereof, thereby effectively treating or preventing the disorder to at least some degree. The term “treating” means at least an amelioration of the symptoms associated with the disorder. The term “amelioration” is used in a broad sense to refer to at least a reduction in the magnitude of a parameter, e.g., symptom, associated with the disorder being treated, such as elevated plasma VLDL or triglyceride levels. As such, “treating” also includes situations where the disorder, or at least symptoms associated therewith, are completely inhibited (e.g., prevented from happening) or stopped (e.g., terminated) such that the subject no longer suffers from the disorder, or at least the symptoms that characterize the disorder (e.g., plasma VLDL and/or triglyceride levels are returned to normal).

The treatment of obesity can be measured as a reduction in the subject's weight, a reduction of the body mass index (BMI), a reduction in the consumption of food intake, and/or a reduction in the desire to consume food. Obesity and overweight generally are defined by BMI in humans, which is correlated with total body fat and serves as a measure of the risk of certain diseases. BMI is calculated by weight in kilograms divided by height in meters squared (kg/m²). Overweight is typically defined as a BMI of 25-29.9 kg/m², and obesity is typically defined as a BMI of 30 kg/m² or higher. Obesity in dogs and cats is usually defined by Body Condition Score (BCS). Obesity is greater than or equal to 8 and overweight is greater than or equal to 6 on a 9-point scale (Purina), or obesity is greater than or equal to 5 and overweight is greater than or equal to 4 on a 5-point scale (Hill's). The 9-point Purina scale is further discussed in Laflamme, Proc. N. Am. Vet. Conf., Jan 16-21, 1993, Orlando, Fla., pp. 290-291; and Laflamme et al., J. Vet. Int. Med., 8: 154 (1994). Overweight and obese subjects typically have an increased risk of developing prediabetes and/or diabetes.

Prediabetes is characterized by impaired fasting glucose (IFG) and/or impaired glucose tolerance (IGT). Most individuals with IFG or IGT will develop progressive hyperglycemia, eventually meeting the criteria for type 2 diabetes. Patients with prediabetes typically have a high cardiovascular disease (CVD) risk as well (e.g., reported hazard ratio 1.1-1.4). IFG is defined by an elevated fasting plasma glucose (FPG) concentration (≧100 and <126 mg/dL). IGT is defined by an elevated 2 hr plasma glucose concentration (≧140 and <200 mg/dL) after a 75 g glucose load on the oral glucose tolerance test (OGTT) in the presence of an FPG concentration <126 mg/dL.

The treatment of hyperlipidemia, arteriosclerosis, coronary artery diseases, and/or metabolic syndrome can be measured as a reduction in lipid and/or cholesterol levels, such as a reduction of at least 15%, at least 25%, at least 30%, at least 40%, at least 50%, at least 60%, at least 70%, at least 75%, or at least 80% compared to control blood levels. The treatment of prediabetes and/or diabetes (e.g., type 2 diabetes) can be measured as maintaining blood glucose levels within acceptable ranges for a given subject. The treatment of hypertension can be measured as maintaining blood pressure within an acceptable range for a given subject.

The compound of formula (I), or a pharmaceutically acceptable salt, solvate, or hydrate thereof, is administered to a subject, such as a mammal. Suitable mammals are from the order Carnivora, including Felines (cats) and Canines (dogs), the order Artiodactyla, including Bovines (cows) and Swines (pigs), the order Perssodactyla, including Equines (horses), the order Primates, Ceboids, or Simioids (monkeys), or the order Anthropoids (humans and apes). An especially preferred mammal is the human. In certain embodiments, the human is an obese human, a human with type 2 diabetes, an obese human with type 2 diabetes, a human with prediabetes, and/or an obese human with prediabetes.

In another embodiment of the invention, the compound of formula (I) is indicated for obesity management, including weight loss and weight maintenance, when used in conjunction with a reduced calorie diet when administered to a patient in need thereof according to one or more methods of the present invention.

In another embodiment of the invention, the compound of formula (I) is indicated to reduce the risk for weight regain after prior weight loss when administered to a patient in need thereof according to one or more methods of the present invention.

In another embodiment, the compound of formula (I) is indicated for an obese patient with an initial BMI≧30 kg/m² or ≧27 kg/m² in the presence of other risk factors (e.g., hypertension, diabetes, dyslipidemia) when administered to a patient in need thereof according to one or more methods of the present invention.

In another embodiment, the compound of formula (I) is indicated as an adjunct to diet and exercise to improve glycemic control in a patient with type 2 diabetes when administered to a patient in need thereof according to one or more methods of the present invention.

In another embodiment, the compound of formula (I) is indicated as an adjunct to diet, exercise, and/or a reduced calorie diet (i) to improve glycemic control, and (ii) for obesity management, including weight loss and weight maintenance, when administered to a patient in need thereof (e.g., an obese type 2 diabetes patient, an obese prediabetes patient) according to one or more methods of the present invention.

In another embodiment of the invention, the compound of formula (I) is indicated to treat IFG and/or IGT when administered to a patient in need thereof (e.g., a prediabetes patient, an obese prediabetes patient) according to one or more methods of the present invention.

In another embodiment of the invention, the compound of formula (I) is indicated to reduce the risk for diabetes when administered to a patient in need thereof (e.g., a prediabetes patient, an obese prediabetes patient, an obese patient) according to one or more methods of the present invention.

As used herein, the term “patient” is a subject, as defined herein. Preferably the patient is a human and can be an infant, child, adolescent, or adult. Preferably, the patient is an adult human.

### EXAMPLES

The following examples further illustrate the invention but, of course, should not be construed as in any way limiting its scope. All studies in the following examples were conducted in accordance with the principles of Good Clinical Practice (GCP).

The following abbreviations are used herein:

AE Adverse event

ALP Alkaline phosphatase

ApoAl Apolipoprotein Al

ApoB Apolipoprotein B

ApoB₄₈ Apolipoprotein B48

ApoCII Apolipoprotein CII

ApoCIII Apolipoprotein CIII

AUC Area under the concentration-time curve

AUC_{0-τ} Area under the curve over the dosing interval

AUCMB Area under the curve minus baseline

BLQ Beneath limit of quantification

BID Twice daily

BMI Body mass index

Cₘₐₓ Maximum concentration

Cₘᵢₙ Minimum concentration

CM-C Chylomicron cholesterol

CM-TG Chylomicron triglyceride

CV Coefficient of variation

GCP Good clinical practice

GI Gastrointestinal

HDL High density lipoprotein

HDL-C High density lipoprotein cholesterol

HDL-TG High density lipoprotein triglyceride

HTG Hypertriglyceridemia

LDL Low density lipoprotein

LDL-C Low density lipoprotein cholesterol

LDL-TG Low density lipoprotein triglyceride

LLOQ Lower limit of quantification

MTP Microsomal triglyceride transfer protein

NC Not calculable

non-HDL Non-high density lipoprotein

QD Once daily

RLP-C Remnant-like particle cholesterol

RLP-TG Remnant-like particle triglyceride

SAE Serious adverse event

SD Standard deviation

sd LDL Small dense LDL cholesterol

SOC System organ class

t_{1/2} Elimination half-life

TG Triglyceride

tₘₐₓ Time to reach the maximum concentration

VLDL Very low density lipoprotein

VLDL-C Very low density lipoprotein cholesterol

VLDL-TG Very low density lipoprotein triglyceride

### Example 1

This example involved a clinical study of the compound of formula (I), which was a single-blind, placebo-controlled, repeated oral dose study to evaluate the safety, tolerability, and pharmacokinetics/pharmacodynamics of the compound of formula (I) administered 5 mg or 15 mg after meals for 14 days twice daily in healthy male subjects.

Eighteen healthy Japanese males 20 to 35 years of age with a BMI of 18.5 to 25 kg/m² participated in the study. The subjects were divided into two dosing groups, and within each dosing group, the subjects were divided as follows: 6 subjects received the compound of formula (1) and 3 subjects received placebo. The dosing regimen was oral administration of a capsule twice a day for 14 days, in which each capsule contained 5 mg (10 mg/day) or 15 mg (30 mg/day). Subjects were in a fed condition prior to administration of each capsule.

Each active capsule contained a filling solution and a gelatin solution. The filling solution comprised the compound of formula (I) (5 mg), propylene glycol esters of fatty acids, and dehydrated ethanol. The gelatin solution comprised purified water, gelatin, and anhydrized liquid sorbitol. The filling solution and gelatin solution were prepared individually and combined to form a soft capsule. The placebo capsules were prepared in a similar manner except that the filling solution contained only propylene glycol esters of fatty acids and dehydrated ethanol.

Physiological examinations (e.g., blood pressure, pulse rate, body temperature, body weight, standard 12-lead electrocardiography (ECG), and ophthalmological test) and laboratory tests (e.g., hematology, blood biochemistry, blood coagulation test) were performed on each subject at each stage of the study (i.e., before the study, after administration of the active or placebo capsules, and after the conclusion of the study). Plasma and urine concentrations of the compound of formula (I) and its metabolite M1 (2-(3-(dimethylcarbamoyl)-4-(4′-(trifluoromethyl)biphenyl-2-ylcarboxamido)phenypacetic acid) were determined by the LC/MS/MS method, with lower limits of quantization of 3.0 ng/mL for plasma concentrations and 6.5 ng/mL for urine concentrations. Adverse event (AE) information was collected from each subject.

Safety

All 18 subjects (i.e., 12 subjects and 6 subjects in the active and placebo groups, respectively) were included in the safety analysis. In this study, neither death nor serious adverse events were reported in any subject. Eleven (92%) of 12 subjects in the active group experienced 44 adverse events. In the 5 mg group, 12 adverse events occurred in 5 (83%) of 6 subjects. In the 15 mg group, 32 adverse events occurred in all 6 subjects (100%). In the placebo group, 3 adverse events occurred in 2 (33%) of 6 subjects. Common adverse events in the active group included “diarrhea” (8/12, 67%), “stomach discomfort” (7/12, 58%), “anorexia” (5/12, 42%), “abdominal discomfort” (3/12, 25%), and “ALT increased” (2/12, 17%). Common adverse events by system organ class were “gastrointestinal disorders” (9/12, 75%), “metabolism and nutrition disorders” (5/12, 42%), and “investigations” (4/12, 33%). Moderate adverse events were noted only in the active group: “diarrhea” (7/12, 58%), “stomach discomfort” (1/12, 8%), “feeling queasy” (1/12, 8%), and “abdominal discomfort” (1/12, 8%). The other adverse events were all graded as mild events.

Based on these findings, “diarrhea” was considered as a noteworthy adverse event in this study. In an earlier phase 1 clinical study involving the administration of a single dose of the compound of formula (I), “diarrhea” was observed at doses of 60 mg or higher, and the incidence was higher in postprandial dose groups. In the study of this example, “diarrhea” occurred at doses of 5 mg or higher, i.e., at lower doses than the doses at which “diarrhea” occurred in the earlier single dose study. This effect was attributed to continuous MTP inhibition in the small intestine with repeated administration of the compound of formula (I). In the 15 mg group, “diarrhea” occurred frequently (all 6 subjects); however, the event was not accompanied by abdominal pain, and these subjects completed the 14-day repeated administration without treatment for diarrhea (albeit under full observation via hospitalization in the study). In consideration of long-term treatment in outpatients, the tolerable dosage is considered as 5 mg BID.

Decreased vitamins A and E were observed in the 15 mg group and appear to be due to the inhibitory effect on lipid absorption by MTP inhibition in the small intestine. In a 3-month oral repeated dose toxicity study of the compound of formula (I) in dogs, vitamins D and E decreased or tended to decrease in dogs given 25 mg/kg of the compound of formula (I).

Pharmacokinetics

the compound of formula (I) was orally administered at 5 or 15 mg BID after meals (specifically, within 180 minutes of the commencement of eating) at intervals of 10 and 14 hours for 14 days. In the 5 mg group, plasma concentrations of the compound of formula (I) were BLQ (<3.0 ng/mL) in all subjects. In the 15 mg group, the compound of formula (I) was detected in plasma in 4 of 6 subjects at 4 hours after the first dose on Day 7 or at 4 hours after the final dose on Day 14. The metabolite M1 (2-(3-(dimethylcarbamoyl)-4-(4′-(trifluoromethyl)biphenyl-2-ylcarboxamido)phenyl)acetic acid) of the compound of formula (I) was detected in plasma of all subjects receiving the compound of formula (I). In the 5 mg group, the mean Cₘᵢₙ for the metabolite M1 was 8.33 ng/mL on Day 2 and elevated to 25.97 ng/mL (3.12 times) on Day 6. After Day 7, the mean Cₘᵢₙ ranged from 22.71 to 27.35 ng/mL (2.73 to 3.28 times). In the 15 mg group, the mean Cₘᵢₙ for the metabolite M1 was 33.57 ng/mL on Day 2 and elevated to 181.90 ng/mL (5.42 times) on Day 8 without a further subsequent increase.

the compound of formula (I) was not excreted in urine in any of the subjects in the 5 or 15 mg group. In contrast, the metabolite M1 was excreted in urine in all subjects in the 5 and 15 mg groups.

MTP inhibitors are considered to suppress chylomicron secretion in the small intestine and inhibit VLDL secretion in the liver. However, no or negligible the compound of formula (I) was detected in the plasma of the subjects. Since the compound of formula (I)'s metabolite M1 (2-(3-(dimethylcarbamoyl)-4-(4′-(trifluoromethy)biphenyl-2-ylcarboxamido)phenyl)acetic acid) has no MTP inhibitory activity, the compound of formula (I) is considered to have reached the small intestine and directly exerted an MTP inhibitory action, with a minor MTP inhibitory activity exerted in the liver.

Serum Lipid Levels

In this study, lipids related to lipoprotein metabolism were determined as serum lipid test items. In the active group, postprandial elevations for RLP-C, TG, LDL-TG, HDL-TG, CM-TG, and ApoB48 (reference data) were inhibited, while LDL-C, HDL-C, TC, and ApoB values decreased. The manifestation of those effects required 3 to 7 days. The compound of formula (I) inhibited an increase in postprandial values for RLP-C, TG, LDL-TG, HDL-TG, and CM-TG after the first dose on Day 1. This effect is attributable to the inhibition of diet-derived lipid absorption based on the MTP inhibitory activity of the compound of formula (I) in the small intestine.

Example 2

This example involved a clinical study of the compound of formula (I), which was a single-blind, placebo-controlled, repeated oral dose study to evaluate the safety, tolerability, and pharmacokinetics/pharmacodynamics of the compound of formula (I) tablets administered for two weeks either once or twice daily in healthy male subjects.

Thirty-two healthy Japanese males 20 to 35 years of age with a BMI of 18.5 to 25 kg/m² participated in the study. The subjects were divided into four dosing groups, and within each dosing group, the subjects were divided as follows: 6 subjects received the compound of formula (I) and 2 subjects received placebo. The dosing regimen was oral administration of a tablet either once (10 mg, 20 mg, or 30 mg) or twice (5 mg) a day for 14 days after breakfast (and after dinner in the case of BID dosing) (specifically, within 180 minutes of the commencement of eating). Each tablet contained 5 mg (BID), 10 mg (QD), 20 mg (QD), or 30 mg (QD).

Each active tablet contained the compound of formula (I) (5 mg or 10 mg), povidone (K30), light anhydrous silicic acid, carmellose calcium, crospovidone, and magnesium stearate. The placebo tablets were prepared in a similar manner except that the tablets did not contain the compound of formula (I).

Physiological examination tests (e.g., blood pressure, pulse rate, body temperature, body weight, standard 12-lead electrocardiography (ECG), and ophthalmological test) and laboratory tests (e.g., hematology, blood biochemistry, blood coagulation test) were performed on each subject at each stage of the study (i.e., before the study, after administration of the active or placebo tablets, and after the conclusion of the study). Plasma and urine concentrations of the compound of formula (I) and its metabolite M1 were determined by the LC/MS/MS method, with lower limits of quantization (LLOQ) of 0.3 ng/mL for plasma concentrations and 6.5 ng/mL for urine concentrations. For the determination of plasma levels of the compound of formula (I) and metabolite M1 in this study, the bioanalytical method was utilized to provide a sensitivity (LLOQ of 0.3 vs. 3 ng/mL). Adverse event (AE) information was collected from each subject.

Safety

All 32 subjects receiving the investigational product were included in the safety analysis. No death or other serious adverse events occurred in the study. No subjects discontinued the study, and no other significant adverse events occurred. Overall, in the compound of formula (I) group, 17 (70.8%) of 24 subjects experienced adverse events. Three (50.0%) of 6 subjects in the 10 mg QD group, 4 (66.7%) of 6 subjects in the 20 mg QD group, 4 (66.7%) of 6 subjects in the 30 mg QD group, and all 6 subjects (100.0%) in the 5 mg BID group experienced adverse events. In the placebo group, 1 (12.5%) of 8 subjects experienced adverse events.

Of adverse events by SOC in the active group, “gastrointestinal disorder” adverse events occurred in 14 (58.3%), “infection and infestation” adverse events occurred in 1 (4.2%), “metabolism and nutrition disorder” adverse events occurred in 1 (4.2%), and “investigation” adverse events occurred in 5 (20.8%) of 24 subjects. At the same daily dosage, the incidence of “gastrointestinal disorder” events was lower in the 10 mg QD group than those in the 5 mg BID group.

Relatively common adverse events observed in at least 3 (12.5%) of 24 subjects in the overall the compound of formula (I) group were “diarrhea” in 11 subjects (45.8%), “stomach discomfort” in 6 subjects (25.0%), “loose stools” in 5 subjects (20.8%), “abdomen enlarged feeling” in 5 subjects (20.8%) and “CK increased” in 3 subjects (12.5%). As moderate adverse events, “diarrhea” was reported in 1 (16.7%) of 6 subjects in the 20 mg QD group, in 3 (50.0%) of 6 subjects in the 30 mg QD group, and in 4 (66.7%) of 6 subjects in the 5 mg BID group. “Vomiting” was observed in 1 (16.7%) of 6 subjects in the 5 mg BID group. The other adverse events were all graded to be “mild” in severity. All of these adverse events did not require any treatments and disappeared subsequently.

Since “diarrhea” was common in the active group, “diarrhea” is considered as a noteworthy adverse event. Twelve events of “diarrhea” were reported in 11 (45.8%) of 24 subjects in the overall the compound of formula (I) group. One event of “diarrhea” in 1 (16.7%) of 6 subjects in the 20 mg QD group, 4 events in 4 (66.7%) of 6 subjects in the 30 mg QD group, and 7 events in all (100.0%) of 6 subjects in the 5 mg BID group. All of these subjects completed study drug administration without any treatments. In 1 subject in the 30 mg QD group and 3 subjects in the 5 mg BID group, the symptom disappeared after the completion of administration. In the remaining 7 subjects, the symptom disappeared during the administration period. In all the subjects who had “diarrhea,” no concomitant symptoms such as abdominal pain appeared, but in 7 of the subjects, dietary intake was reduced (extent of complete intake <90%).

In the 30 mg QD group, vitamin E levels in the subjects decreased on Day 7. A further decrease in vitamin E levels was not seen on Day 15. In the remaining groups, fat-soluble vitamin levels in the subjects did not decrease. The decreased vitamin E level in the subjects of the 30 mg QD group was believed to have been induced by the pharmacological action of the compound of formula (I) in the small intestine. In contrast, decreases in the levels of vitamins A and E were observed in the 15 mg BID group in the phase I repeated dose (capsules) study (Example 1).

In the 14-day repeated dose treatment of the compound of formula (I), “diarrhea” occurred in 4 of 6 subjects at the 30 mg dose. Symptoms disappeared in 3 subjects during the administration period and the remaining 1 subject after the final administration day without any treatments. “Queasiness” occurred in 2 of 6 subjects. “Queasiness” and other adverse events disappeared until the final administration day without any treatments. At the 20 mg dose, “diarrhea” occurred in 1 of 6 subjects and disappeared during the administration period without any treatments.

In the 14-day repeated dose treatment at a dose of 5 mg BID, “diarrhea” occurred in 6 of 6 subjects. Symptoms disappeared in 3 subjects during the administration period and the remaining 3 subjects after the final administration day without any treatments. “Vomiting” occurred in 1 of 6 subjects. “Vomiting” and other adverse events disappeared until the final administration day without any treatments. Taking into consideration of the incidence of “diarrhea,” 5 mg BID was judged to be inappropriate as a clinical dose for hyperlipidemic patients.

Pharmacokinetics

After the initial dose on Day 1, the plasma the compound of formula (I) was quantifiable in all the compound of formula (I) groups. In the 10 mg QD group, Cₘᵢₙ of the compound of formula (I) was below the limit of quantization on Day 2 to 14. In the 20 mg QD, 30 mg QD, and 5 mg BID groups, Cₘᵢₙ of the compound of formula (I) was detected sporadically in 1, 4, and 4 of each 6 subjects, respectively. However, in all subjects, Cₘᵢₙ of the compound of formula (I) was less than 3 ng/mL (i.e., less than the LLOQ).

In the 10 mg QD, 20 mg QD and 30 mg QD groups, Cₘₐₓ of the compound of formula (I) after the initial dose on Day 1 tended to increase in a greater than dose-proportional manner. AUC₀₋₂₄ₕᵣ of the compound of formula (I) after the initial dose on Day 1 was considered to increase in a greater than dose-proportional manner. Cₘₐₓ and AUC₀₋₂₄ₕᵣ of the compound of formula (I) after the final dose on Day 14 were considered to increase in a dose-proportional manner.

After the initial dose on Day 1, plasma metabolite M1 was quantifiable in all the compound of formula (I) groups. Cₘᵢₙ of the metabolite M1 was quantifiable in all the compound of formula (I) groups on Day 2 to 14. An increase in Cₘᵢₙ on and after Day 3 varied 1.3 to 2.8-fold compared to Cₘᵢₙ on Day 2. Large inter-subject variation and intra-subject variation were observed.

In the 10 mg QD, 20 mg QD and 30 mg QD groups, Cₘₐₓ and AUC₀₋₂₄ₕᵣ of the metabolite M1 after the initial dose on Day 1 were considered to increase in a greater than a dose-proportional manner. However Cₘₐₓ and AUC₀₋₂₀, of the metabolite M1 after the final dose on Day 14 were considered to increase in a dose-proportional manner. The compound of formula (I) was not quantifiable in urine in all the compound of formula (I) groups. The metabolite M1 was excreted in urine in all the compound of formula (I) groups. Urinary excretions of metabolite M1 were similar among the active groups. However, the cumulative percent of dose excreted in urine of metabolite M1 had large inter-subject variation, and the daily percent of dose excreted in urine of metabolite M1 had large intra-subject variation during multiple dosing.

Serum Lipid Levels

In the active group, the expected postprandial elevations for TG, HDL-TG, LDL-TG, Midband-TG, VLDL-TG, CM-TG, and ApoB48 were suppressed, and TC, HDL-C, LDL-C, and ApoB values decreased by and after Day 7. The suppression of TG, HDL-TG, LDL-TG, Midband-TG, VLDL-TG, and CM-TG postprandial elevation was considered to be attributable to the suppression of lipid absorption by MTP inhibition in the small intestine. The decreased TC, HDL-C, and LDL-C were considered to be attributable to not only the suppression of lipid absorption by MTP inhibition in the small intestine, but also diarrhea and reduced dietary intake.

### Example 3

This example involved a clinical study of the compound of formula (I), which was a repeated dose study of tablets.

Eighteen healthy Japanese males 20 to 35 years of age with a BMI of 18.5 to 25 kg/m² participated in the study. The subjects were divided into two dosing groups and within each dosing group, the subjects were divided as follows: 6 subjects received the compound of formula (I) and 3 subjects received placebo. The dosing regimen was oral administration of a tablet twice a day for 14 days after breakfast and dinner (specifically, within 180 minutes of the commencement of eating), in which each tablet contained 1 mg (2 mg/day) or 2 mg (4 mg/day).

Each active tablet contained the compound of formula (I) (1 mg or 2 mg), povidone (K30), light anhydrous silicic acid, carmellose calcium, crospovidone, and magnesium stearate. The placebo tablets were prepared in a similar manner except that the tablets did not contain the compound of formula (I).

Physiological examinations (e.g., blood pressure, pulse rate, body temperature, body weight, standard 12-lead electrocardiography (ECG), and ophthalmological test) and laboratory tests (e.g., hematology, blood biochemistry, blood coagulation test) were performed on each subject at each stage of the study (i.e., before the study, after administration of the active or placebo tablets, and after the conclusion of the study).

Plasma and urine concentrations of the compound of formula (I) and its metabolite M1 were determined by the LC/MS/MS method, with lower limits of quantization (LLOQ) of 0.3 ng/mL for plasma concentrations and 6.5 ng/mL for urine concentrations. For the determination of plasma concentrations of the compound of formula (I) and metabolite M1 in this study, the bioanalytical method achieved a sensitivity (LLOQ) of 0.3 ng/mL. Adverse event (AE) information was collected from each subject.

Safety

All 18 subjects were included in the safety analysis. No death or other serious adverse events occurred in the study. No adverse events resulted in discontinuation of administration of the compound of formula (I) or the placebo or of observation after administration, and no other significant adverse events occurred. Overall, in the active group, seven (58.3%) of 12 subjects experienced adverse events. In particular, four (66.7%) of six subjects in the 1 mg BID group and three (50.0%) of six subjects in the 2 mg BID group experienced adverse events. In the placebo group, no subjects experienced adverse events. Adverse events by SOC in the overall the compound of formula (I) group included “gastrointestinal disorders” in five (41.7%) and events of “investigations” in four (33.3%) of 12 subjects. Relatively common adverse events observed in at least two (16.7%) of 12 subjects in the overall the compound of formula (I) group were “diarrhea” in three subjects (25.0%) and “loose stools” in three subjects (25.0%). As adverse drug reactions, gastrointestinal disorders (diarrhea and loose stools) and events of investigations (increased alkaline phosphatase and triglyceride) occurred. All the adverse events including adverse drug reactions reported in the study were mild and disappeared without any treatment.

In the previous phase I study of the compound of formula (I) tablets (Example 2), diarrhea occurred in six (100%) of six subjects and was assessed to be “moderate” in four (66.7%) of six subjects in the 5 mg BID group. In the present study, the incidence of diarrhea was low, and all the diarrhea events were assessed to be “mild”. In the previous phase I study (Example 2), vitamin E levels decreased in the 30 mg QD group. In the present study, no decrease in fat-soluble vitamins (vitamin A, 25-OH vitamin D, and vitamin E) was observed. In this study, 14-day repeated dose treatment was completed in all of the dose groups. All the adverse events after repeated-dose administration of the compound of formula (I) tablets twice daily for 14 days were mild and disappeared without any treatment. Based on this finding, the compound of formula (I) tablets are considered tolerable at doses of up to 2 mg when administered twice daily for 14 days.

Pharmacokinetics

In all the dose groups, plasma concentrations of the compound of formula (I) were below the limit of quantization. Plasma metabolite M1 was quantifiable in both the active groups. Cₘᵢₙ of the metabolite M1 on and after Day 5 was 3.5 to 4.9 times and 2.7 to 3.7 times as high as those on Day 2 in the 1 and 2 mg BID groups, respectively. Although attainment of a steady state could not be determined because of large inter-subject and intra-subject variability in plasma concentrations of the metabolite M1 and intra-subject variations in the daily concentration, Cₘᵢₙ of the metabolite M1 on and after Day 5 generally appeared to range from 4.35 to 6.02 ng/mL in the 1 mg BID group and from 7.27 to 10.08 ng/mL in the 2 mg BID group.

Although no differences were found between the doses in the urinary excretion of the metabolite M1, the cumulative percent of dose excreted in urine of the metabolite M1 had large inter-subject variabilities, and the daily percent of dose excreted in the urine of metabolite M1 had large intra-subject variabilities.

Serum Lipid Levels

In the active groups, the drug inhibited a postprandial elevation in TG, HDL-TG, LDL-TG, Midband-TG, and CM-TG. The suppression of TG, HDL-TG, LDL-TG, Midband-TG, and CM-TG postprandial elevation was considered to be attributable to the suppression of diet-derived lipid absorption by MTP inhibition in the small intestine.

### Example 4

This example involved a clinical study of the compound of formula (I), which was a randomized, parallel group study evaluating the efficacy of the compound of formula (I) administered twice daily for eight weeks in Japanese subjects with hyperlipidemia.

One hundred five Japanese patients with hyperlipidemia (LDL-C between 140 and 220 mg/dL; TG≦400 mg/dL) participated in the study. The subjects were divided into four dosing groups, and within each dosing group, the subjects were divided as follows: 25 subjects received 0.5 mg BID, 28 subjects received 1 mg BID, 25 subjects received 2 mg BID, and 27 subjects received 4 mg BID. The dosing regimen was oral administration of a capsule twice a day for 8 weeks after breakfast and dinner (specifically, within 180 minutes of the commencement of eating), in which each capsule contained 0.5 mg, 1 mg, 2 mg, or 4 mg. For the efficacy analysis, 95 subjects were observed (0.5 mg BID: 25 subjects, 1 mg BID: 24 subjects, 2 mg BID: 22 subjects, 4 mg BID: 24 subjects).

Each active capsule contained a filling solution and a gelatin solution. The filling solution comprised the compound of formula (I) (0.5 mg, 1 mg, 2 mg, or 4 mg), propylene glycol esters of fatty acids, and dehydrated ethanol. The gelatin solution comprised purified water, gelatin, and anhydrized liquid sorbitol. The filling solution and gelatin solution were prepared individually and combined to form a soft capsule. The placebo capsules were prepared in a similar manner except that the filling solution contained only propylene glycol esters of fatty acids and dehydrated ethanol.

The objective was to administer the compound of formula (I) orally to hyperlipidemic patients at a dose of 0.5, 1, 2, or 4 mg as capsules twice daily after the morning (e.g., breakfast) and evening meal (e.g., dinner) for eight weeks in order to explore a dose range in which the compound of formula (I) reduced LDL-C levels. In addition, the effects on lipid metabolism, safety, and pharmacokinetics of the compound of formula (I) were evaluated.

Physiological examinations (e.g., blood pressure, pulse rate, body temperature, body weight, standard 12-lead electrocardiography (ECG), and ophthalmological test) and laboratory tests (e.g., hematology, blood biochemistry, blood coagulation test) were performed on each subject at each stage of the study (i.e., before the study, after administration of the active or placebo capsules, and after the conclusion of the study). Plasma and urine concentrations of the compound of formula (I) and its metabolite M1 were determined by the LC/MS/MS method, with the lower limit of quantization of 3.0 ng/mL for plasma concentrations and 6.5 ng/mL for urine concentrations. Adverse event (AE) information was collected from each subject.

Primary Endpoint

The mean percent change in LDL-C levels at Week 8 from baseline, which was assessed as the primary efficacy endpoint, was −4.3% in the 0.5 mg BID group, −4.5% in the 1 mg BID group, −12.2% in the 2 mg BID group, and −12.3% in the 4 mg BID group. In the 2 and 4 mg BID groups, mean percent changes in LDL-C levels at Week 8 represented a statistically significant reduction in the levels from baseline.

Secondary Endpoints

TC levels decreased throughout the treatment period as with LDL-C levels in the 2 and 4 mg BID groups, whereas no definite trend or dose correlation was seen in the time course of TG or HDL-C levels during the treatment period.

Among apolipoproteins, ApoB and ApoE levels decreased throughout the treatment period, as with LDL-C levels, in the 2 and 4 mg BID groups, whereas no significant changes were found in ApoB48, ApoA-I, ApoA-II, ApoC-II, and ApoC-III levels during the treatment period.

Time course of mean LDL-C and HDL-C fraction levels and those of mean percent changes in the values determined with electrophoresis were similar to those for LDL-C and HDL-C levels determined with an enzymatic assay (direct method). Apart from LDL-C fraction level, no dose correlation was found in changes in HDL-C fraction, VLDL-C, Midband-C, CM-C, HDL-TG, VLDL-TG, Midband-TG, LDL-TG, or CM-TG level during the treatment period.

Other Endpoints

The compound of formula (I) did not affect the insulin, hemoglobin Alc, adiponectin, or leptin level.

Safety

All of the 105 subjects treated with the compound of formula (I) were included in the safety analysis. In this study, neither death nor other serious adverse events occurred. At the discretion of the investigator, four subjects were withdrawn from the study treatment because of adverse events. Nausea in one subject in the 1 mg BID group and diarrhea in two subjects in the 4 mg BID group were classified into gastrointestinal disorders, and their casual relationship to the investigational product cannot be ruled out (adverse drug reactions). Of the 105 subjects treated with the compound of formula (I), 75 subjects (71.45%) experienced 213 adverse events. The number of subjects with any adverse events (incidence) and that of adverse events by dose groups were as follows: 20 events in 11 (44.0%) of 25 subjects in the 0.5 mg BIG group; 60 events in 21 (75.0%) of 28 subjects in the 1 mg BID groups; 51 events in 20 (80.0%) of 25 subjects in the 2 mg BID groups; and 82 events in 23 (85.2%) of 27 subjects in the 4 mg BID group.

When adverse events were analyzed by SOC, the highest incidences of “gastrointestinal disorders” in the 105 subjects were as follows: 117 events of “gastrointestinal disorders” in 50 subjects (47.6%); 49 events of “investigations” in 33 subjects (31.4%); 11 events of “infections and infestations” in 10 subjects (9.5%); 7 events of “musculoskeletal, and connective tissue disorders” in 7 subjects (6.7%); and 7 events of “metabolism and nutrition disorders” in 7 subjects (6.7%). Among gastrointestinal disorders, diarrhea occurred frequently. The number of diarrhea events is follows: 6 events in 5 (15.9%) of 28 subjects in the 1 mg BID groups; 12 events in 6 (24.0%) of 25 subjects in the 2 mg BID group; and 21 events in 11 (40.7%) of 27 subjects in the 4 mg BID group.

Twenty-six adverse events were classified as “moderate” in 19 (18.1%) of 105 subjects treated with the compound of formula (I). Diarrhea only was reported as a moderate adverse event when it occurred in two or more subjects. The number of subjects with moderate diarrhea (incidence) and that of moderate diarrhea events by dose group were as follows: 2 events in 2 (8.0%) of 25 subjects in the 0.5 mg BID group; 0 event in 0 (0.0%) of 28 subjects in the 1 mg BID group; 4 events in 3 (12.0%) of 25 subjects in the 2 mg BID group; and 13 events in 8 (29.6%) of 27 subjects in the 4 mg BID group.

With regard to laboratory test values, laboratory abnormalities in 3 (2.9%) or more of 105 subjects treated with the compound of formula (I) were CPK in 7 subjects (6.7%), ALT in 6 subjects (5.7%), CRP in 4 subjects (3.8%), γ-GTP in 4 subjects (3.8%), and IP in 3 subjects (2.9%). Laboratory abnormalities in 2 or more subjects in each dose group were as follows: ALT, CPK, CRP, and urine ketone each in 2 subjects in the 4 mg BID group; ALT, γ-GTP, and IP each in 2 subjects in the 2 mg BID group; ALT and CPK each in 2 subjects in the 1 mg BID group; and CPK in 3 subjects in the 0.5 mg BID group.

For fat-soluble vitamins (vitamin A, 25-OH vitamin D, and vitamin E), no clinically significant changes were noted.

Pharmacokinetics

Plasma the compound of formula (I) concentrations were not quantifiable (i.e., below the lower limit of quantification of 0.3 ng/mL) in the 0.5 and 1 mg BID dose groups. In the 2 and 4 mg BID groups, the compound of formula (I) concentrations also were not quantifiable, except in a few sporadic cases in which levels close to the lower limit of quantification were observed.

The Cₘᵢₙ values of metabolite M1 (Week 1 to Week 8) ranged from 2.61 to 2.95 ng/mL in the 0.5 mg BID group, 4.34 to 5.32 ng/mL in the 1 mg BID group, 10.69 to 12.40 ng/mL in the 2 mg BID group, and 23.42 to 25.40 ng/mL in the 4 mg BID group. Cₘᵢₙ values increased with dose and, within each dose group, the values did not change markedly from Week 1 to Week 8.

In all of the dose groups, Cₘᵢₙ values of metabolite M1 tended to be higher in females than in males. However, body weight adjusted values were similar, indicating that body weight differences between females and males may be one of factors in the gender differences in the observed Cₘᵢₙ values.

### Example 5

This example involved a clinical study of the compound of formula (I), which was an investigation of efficacy and safety by administration at a fixed-dose or escalating doses.

Fifty-two Japanese patients with hyperlipidemia (LDL-C between 140 and 220 mg/dL; TG≦400 mg/dL) participated in the study. The subjects were divided into two dosing groups, and within each dosing group, the subjects were divided as follows: 26 were in the fixed-dose group, and 26 subjects were in the dose escalation group. The dosing regimen was oral administration of a tablet twice a day for 12 weeks after breakfast and dinner (specifically, within 180 minutes of the commencement of eating). One group received a fixed dose of 2 mg per tablet through the entire study. The other group received escalating doses of 0.5 mg (weeks 1-4), 1 mg (weeks 5-8), and 2 mg (weeks 9-12). For the efficacy analysis, 50 subjects were observed (25 in the fixed-dose group and 25 in the dose escalation group).

Each active tablet contained the compound of formula (I) (0.5 mg, 1 mg, or 2 mg), povidone (K30), light anhydrous silicic acid, carmellose calcium, crospovidone, and magnesium stearate.

The objective of this study was preliminary evaluation of the efficacy (based on serum lipid levels), safety, and pharmacokinetics of the compound of formula (I) administered orally twice daily after breakfast and supper for 12 weeks as a fixed dose (4 mg/day) or escalating dose (1 mg/day, 2 mg/day and 4 mg/day) regimen in patients with hyperlipidemia.

Efficacy

The LDL-C level in the fixed-dose group showed a maximum decrease (−6.38%) on the observation day of Week 2, and then the change remained in the range of −1.96 to −5.98% through Week 12. The LDL-C level decrease in the dose escalation group was more gradual and reached −7.24% and −7.12% on the observation days of Weeks 10 and 12 (4 mg/day). The change in TC was lower than the change in LDL-C. In the fixed-dose group, a decrease of nearly the maximum was observed on the observation day of Week 2. In the dose escalation group, a dose-dependent decrease was observed. TG level in both groups tended to increase, and fluctuated over the course of the study. TG level in the dose escalation group was maintained at a higher level than that observed in the fixed-dose group throughout the treatment period.

Safety

All 52 subjects receiving the study drug were included in the safety analysis. No death or other serious adverse events occurred in the study. There was one subject who discontinued the study due to an adverse event. This subject was in the fixed-dose group and exhibited a decrease in high-density lipoprotein. The causal relationship to the study drugs was judged as “Related” because no other factors were specified and the value returned to a level similar to baseline level after discontinuation in the study. There was no other severe adverse event. Moderate adverse events occurred in four (15.4%) of 26 subjects in each of the fixed-dose group and the dose escalation group. Of the moderate adverse events, diarrhea was observed in four subjects in the fixed-dose group and in three subjects in the dose escalation group. The number of subjects with adverse events (incidence) and the number of adverse events are as follows: 17 (65.4%) of 26 subjects experienced 45 adverse events in the fixed-dose group; 17 (65.4%) of 26 subjects experienced 37 adverse events in the dose escalation group. Adverse events by SOC in the fixed-dose group were 25 events of “gastrointestinal disorders” in 13 (50.0%) of 26 subjects, nine events of “laboratory test” in six (23.1%) of 26 subjects, and four events of “infections and infestations” in four (15.4%) of 26 subjects. In the dose escalation group, adverse events by SOC were 14 events of “gastrointestinal disorders” in nine (34.6%) of 26 subjects, 11 events of “laboratory test” in six (23.1%) of 26 subjects, and 6 events of “infections and infestations” in five (19.2%) of 26 subjects. In both groups, the incidence of “gastrointestinal disorders” or diarrhea was highest on the observation days of Weeks 1 to 4.

During the observation days of Weeks 1 to 4 and Weeks 5 to 8, the incidence of “gastrointestinal disorders” was lower in the dose escalation group (1 mg/day and 2 mg/day) than in the 4 mg/day of the fixed-dose group. The incidence of “gastrointestinal disorders” was lower in the dose escalation group (20.0%) during the observation days of Weeks 9 to 12 (4 mg/day) than the incidence in the fixed-dose group (46.2%) during the observation days of Weeks 1 to 4 (4 mg/day). In the fixed-dose group, the incidence of “gastrointestinal disorders” decreased with time, and there was only one subject who experienced diarrhea during the observation days of Weeks 9 to 12. These findings suggest the following relationship between the administration method and the occurrence of “gastrointestinal disorders” including diarrhea. By decreasing the starting dose of the compound of formula (I) from 4 mg/day to 1 mg/day, the occurrence of “gastrointestinal disorders” in the early phase of treatment (weeks 1-4) may be reduced. Although the incidence of “gastrointestinal disorders” as adverse events is higher in the early phase of treatment, the incidence appeared to decrease over the course of time. By gradually escalating the dose of the compound of formula (I) from 1 mg/day to 4 mg/day the incidence of “gastrointestinal disorders” may be reduced as compared with the incidence in subjects treated at 4 mg/day from the start.

In clinical laboratory tests, abnormal changes observed in at least two of 52 subjects were “specific gravity urine increased,” “CRP quantitative increased,” “γ-GTP increased,” “CK (CPK) increased,” and “urinary pH increased.” All of these changes were slight changes, showing no trend toward worsening with continuation of treatment. Therefore, these changes were considered unlikely to lead to the development of serious adverse events.

No clinically relevant abnormal changes were observed in systolic blood pressure, diastolic blood pressure, pulse rate, body temperature, body weight, and ultrasonography. Adverse events observed in the standard 12-lead electrocardiogram were “electrocardiogram ST segment depression” in one subject of the fixed-dose group and “electrocardiogram QT corrected interval prolonged” in one subject of the dose escalation group. Both events were mild in severity. These events were judged as “Not related,” because they appeared to be transient events, having resolved without any treatment during the administration of the study drug.

Pharmacokinetic

The Cₘᵢₙ of the compound of formula (I) was obtained only in two subjects at a total of three time points. In other subjects, the Cₘᵢₙ was below the lower limit of quantization on any of the observation days. In the fixed-dose group, the mean Cₘᵢₙ of metabolite M1 was 10.74 to 12.29 ng/mL, and the Cₘᵢₙ of metabolite M1 during the observation day of Weeks 2 to 12 remained comparable. On the other hand, in the dose escalation group, the Cₘᵢₙ of metabolite M1 increased with a dose-proportional manner. The mean Cₘᵢₙ values of metabolite M1 in the fixed-dose group and the dose escalation group were 12.20 ng/mL and 12.15 ng/mL, respectively, on the observation day of Week 10, and were 10.74 ng/mL and 13.47 ng/mL, respectively, on the observation day of Week 12. Thus, the Cₘᵢₙ of metabolite M1 at the same dose level was similar between the two groups. The mean Cₘᵢₙ in the fixed-dose group was 8.98 to 13.10 ng/mL in males and 10.87 to 12.47 ng/mL in females. The mean Cₘᵢₙ on the observation days of Weeks 10 and 12 in the dose escalation group treated at the same dose (4 mg/day) was 12.28 and 10.76 ng/mL, respectively, in males and 12.06 and 15.28 ng/mL, respectively, in females. The Cₘᵢₙ of metabolite M1 was similar between males and females in both groups.

### Example 6

This example involved a clinical study of the compound of formula (I), which was a randomized, double-blind, placebo-controlled, ascending dose study evaluating the safety, tolerability and pharmacokinetics/pharmacodynamics of the compound of formula (I) administered for two weeks either once or twice daily in healthy male subjects and male patients with elevated triglycerides.

Originally, the study was planned as 4 sequential cohorts of healthy male Caucasian subjects (Cohorts 1 to 4) followed by a cohort of male Caucasian patients with elevated triglycerides (HTG patients, Cohort 5). An additional cohort of healthy male Caucasian subjects (Cohort 4.1) was enrolled into the study (6 cohorts in total). The cohorts were as follows: Cohorts 1 to 4.1 had healthy Caucasian males, aged 18-45 years with a body weight of 60-100 kg and body mass index (BMI) between 19-28 kg/m² at screening and fasting triglycerides less than 1.69 mmol/L (less than 150 mg/dL) at the screening visit and Day-3. Cohort 5 had Caucasian males, aged 18-50 years with a body weight of 60-115 kg and BMI less than 35 kg/m² at screening and with elevated fasting triglycerides (TG≧1.69 mmol/L and <5.62 mmol/L; ≧150 mg/dL and <500 mg/dL) at the screening visit and Day-3.

The planned treatment for Cohorts 1 to 4 was twice daily administration of 5 mg, 10 mg or 15 mg the compound of formula (I)/placebo, or once daily administration of 30 mg the compound of formula (1)/placebo. Following treatment administration to Cohort 1, it was determined that twice daily dosing of 5 mg the compound of formula (I) was poorly tolerated; 5 subjects were withdrawn from dosing due to persistent adverse events of moderate intensity (e.g., abdominal pain, abdominal cramps, nausea, loose stools, and decreased appetite). As only the compound of formula (I) 5 mg capsules were supplied for this study, doses were administered once daily to determine the maximum tolerated dose, and the planned doses for each subsequent cohort were amended based on the safety and tolerability data obtained from the preceding cohort. The dose level and dosing schedule planned for the patients in Cohort 5 was determined by the maximum tolerated dose and dosing schedule as reported from the planned Cohorts 1 to 4.1.

Twelve healthy male Caucasian subjects were enrolled in each of Cohorts 1 to 4.1; dose cohorts were randomized in a ratio 9:3 to receive the compound of formula (I) or placebo, respectively, at the dose level associated with that cohort. Twelve male Caucasian patients with elevated triglycerides were randomized in a ratio of 8:4 to receive the compound of formula (I) or placebo, respectively, at the maximum tolerated dose level and dosing schedule.

Each active capsule contained a filling solution and a gelatin solution. The filling solution comprised the compound of formula (I) (5 mg), propylene glycol esters of fatty acid, and dehydrated ethanol. The gelatin solution comprised purified water, gelatin, and anhydrized liquid sorbitol. The filling solution and gelatin solution were prepared individually and combined to form a soft capsule. The placebo capsules were prepared in a similar manner except that the filling solution contained only propylene glycol esters of fatty acids and dehydrated ethanol.

Safety

This study was conducted to investigate the safety and tolerability of the compound of formula (I) and to determine the pharmacokinetic and pharmacodynamic profiles of the compound of formula (I) administered, either once or twice a day, for 14 days to healthy non-fasting male Caucasian subjects and male Caucasian HTG patients. The planned treatment for Cohorts 1 to 4 was twice daily administration of 5 mg, 10 mg or 15 mg the compound of formula (I)/placebo, or once daily administration of 30 mg the compound of formula (I)/placebo. Following Cohort 1, it was determined that twice daily dosing of 5 mg the compound of formula (I) was poorly tolerated. Consequently, doses were administered once daily to healthy volunteers, and the planned doses for each subsequent cohort were amended based on the safety and tolerability data obtained from the preceding cohort.

Although 5 mg, administered twice daily, was poorly tolerated by subjects in Cohort 1, the same dose level and dosing schedule was reasonably well tolerated in Cohort 5 (HTG Patients). It was thought that subjects in Cohort 1 may have been anxious and, with little experience of the effect of the compound of formula (I), there was less confidence that symptoms would resolve spontaneously. This may have led to relative over-reporting of adverse events in Cohort 1.

For healthy subjects in Cohort 1 (5 mg, BID), the compound of formula (I) was not well tolerated, and 5 subjects were withdrawn for adverse events of abdominal cramps, nausea, and syncope (one subject). Gastrointestinal adverse events were more common in the withdrawn subjects from Cohort 1 than in subjects in other cohorts. For healthy subjects in Cohorts 2 (5 mg, QD), 3 (10 mg, QD), 4 (15 mg, QD), and 4.1 (20 mg, QD) the compound of formula (I) was reasonably well-tolerated by the majority of subjects. A total of 178 treatment-emergent adverse events were reported by 46 (77%) of the 60 subjects participating in the study, of whom 34 received the compound of formula (I) (76% of treatment group) and 12 received placebo (80% of treatment group). The most common adverse events were gastrointestinal disorders (nausea and abdominal pain).

For HTG patients in Cohort 5, the compound of formula (I) was reasonably well tolerated by the majority of patients. A total of 48 treatment-emergent adverse events were reported by 11 of the 12 subjects (92%) participating in the study, of whom 7 received the compound of formula (I) (88% of treatment group) and 4 received placebo (100% of treatment group). The most common adverse events were gastrointestinal disorders.

There were no clinically relevant findings in any of the safety analyses in any cohort, which included:

Liver MRIs: these were performed as it had been previously shown that MTP inhibitors may be associated with fatty liver.

Tryptase measurements: these were performed as a measure of allergic reaction, since there was concern over the possibility of covalent bonding, which was seen in pre-clinical studies.

Fat soluble vitamin measurements: these were performed as abetalipoprotenemic patients (deficient in liver and gastrointestinal MTP) have demonstrated malabsoption of fat-soluble vitamins and specifically vitamins A and E.

Fecal fat measurements: these were performed as steatorrhoea was predicted for this type of compound.

Pharmacokinetic

Due to sporadic glycosuria detected in urine collected for pharmacokinetic analysis, additional renal function tests were performed which included creatinine (serum and urine), glucose (serum and urine), retinol binding protein (urine), N-acetyl-beta-D-glucosaminidase (urine), urinary microalbumin, and urine microscopy. Review of these data did not suggest drug-related nephrotoxicity or hyperglycaemia.

Following the first administration and repeated once- and twice-daily oral administration of the compound of formula (I) at 5 to 20 mg to healthy male subjects, plasma concentrations of the compound of formula (I) were generally below the limit of quantification, BLQ (<3 ng/mL). Following the first administration and repeated once- and twice-daily oral administration of the compound of formula (I) at 5 mg to healthy male subjects, plasma concentrations of metabolite M1 were generally only quantifiable in a small number of samples on Day 1 and up to 72 h post-dose on Day 14. Following the first administration and repeated once-daily oral administration at 10 to 20 mg, plasma concentrations of metabolite M1 were generally quantifiable up to at least 24 h post-dose on Day 1, and up to 72 h post-dose on Day 14, indicating continuous exposure to metabolite M1 at the higher dose levels.

Following the first administration and repeated twice-daily administration of the compound of formula (I) at 5 mg to healthy male subjects, maximum plasma concentrations of metabolite M1 were reached (tₘₐₓ) at approximately 3.5 to 4 h post-dose (median estimates). Thereafter, plasma metabolite M1 concentrations declined with a mean apparent terminal half-life of approximately 27 h. Following the first administration and repeated once-daily administration of the compound of formula (I) at 5 to 20 mg to healthy male subjects, metabolite M1 tₘₐₓ was approximately 4 to 5 h post-dose and 5.5 to 11.5 h post-dose (median estimates), respectively. Thereafter, plasma metabolite M1 concentrations declined with a mean apparent terminal half-life of approximately 24 to 42 h. Following the first administration and repeated once-daily oral administration of the compound of formula (I) at 5 to 20 mg to healthy male subjects, the extent of systemic exposure to metabolite M1 appeared to increase in a greater than dose-proportional manner. Furthermore, the 95% CIs for the exponent excluded unity for AUC_{0-τ}, on Day 1 and for AUC_{0-τ}and Cₘₐₓ on Day 14, indicating that the observed deviation from dose-proportionality was statistically significant.

The extent of systemic availability (AUC_{0-τand C}ₘₐₓ) of metabolite M1 after oral administration of 5 mg the compound of formula (I) to patients with elevated triglyceride levels (Cohort 5) appeared to be greater than that in subjects with normal triglyceride levels (Cohort 1). As observed in healthy subjects, plasma concentrations of metabolite M1 in patients were generally only quantifiable in a small number of samples on Day 1 and up to at least 48 h post-dose on Day 14. Maximum plasma concentrations of metabolite M1 in patients were reached at approximately 2 to 5 h post-dose and declined with a mean apparent terminal half-life of approximately 30 h. Following once- and twice-daily oral administration of the compound of formula (I) at 5 to 20 mg to healthy male subjects and patients, plasma concentrations of metabolite M1 tended to reach steady-state by approximately Day 8. Accumulation of metabolite M1 in plasma after repeated, once- and twice-daily oral doses was consistent with time-invariant kinetics.

Following the first administration of the compound of formula (I), urinary recovery of metabolite M1 was limited, representing, on average, less than 8% of the administered dose, with maximum recovery attained by approximately 4 to 5 h post-dose. Following administration of the compound of formula (I) for 14 days, urinary recovery of metabolite M1 up to 72 h post-last dose represented, on average, up to 20% of the total administered dose (adjusted for molecular weight). This increase in urinary recovery is consistent with the observed accumulation of metabolite M1 in plasma over the 14 day dosing period.

Following the first and last, once or twice daily, oral dose of the compound of formula (I), renal clearance of metabolite M1 was estimated to be, on average, approximately 50 to 72 mL/min, which is low compared to glomerular filtration rate in man. The compound of formula (I) suppressed the expected postprandial increase of TG and chylomicron-TG in healthy subjects and patients with high TG. The pharmacological effect of the compound of formula (I) did not appear to demonstrate accumulation or reduction of effect over the 14 day treatment period.

Serum Lipid Levels

Overall, subjects on placebo maintained relatively stable plasma cholesterol levels over the 14 day treatment period. Mean fasting HDL-C decreased 11.63%, and fasting TG levels fell an average of 11.4%, but there was little change in the mean daily fed levels over the 14 day treatment period. The once-daily regimen had no discernible impact on triglyceride-rich lipoproteins as indicated by VLDL indices (ApoCII, ApoCIII, VLDL-TG, VLDL-C). LDL, total cholesterol, and ApoB results suggest a modest, non-dose related effect of the compound of formula (I) on the IDL/LDL range of particles. The 5 mg BID regimen appeared to have more marked effects on LDL-C, HDL-C, VLDL-C, and VLDL-TG than the QD regimens.

The decrease in HDL-C demonstrated in subjects and patients randomized to placebo and active treatment is likely to be related to changes in dietary (e.g., higher carbohydrate and lower alcohol intake levels) and exercise patterns.

### Example 7

This example involved a clinical study of the compound of formula (I), which was a randomized, double-blind, placebo-controlled, ascending dose study to evaluate the safety, tolerability and pharmacokinetics/pharmacodynamics of the compound of formula (I) tablets administered twice daily for two weeks in obese subjects.

A total of 49 subjects were enrolled in this study with all subjects receiving at least one dose of study medication. The subjects were obese Caucasian males, aged 18 to 65 years, with a body mass index (BMI) of ≧30 kg/m² to <40 kg/m² and TG of <500 mg/dL. The subjects were separated into four arms of nine (one with ten) test subjects (which were administered the compound of formula (I)) and three placebo subjects. Each test subject received a tablet taken orally over a period of 14 days after breakfast and dinner (BID) (specifically, within 180 minutes of the commencement of eating) of 1 mg, 2.5 mg, 3.5 mg, or 5 mg, using one or multiples of the 1 mg and 2.5 mg tablets of the compound of formula (I) to achieve the total dose. A total of 40 subjects completed the study according to the protocol. A total of 7 subjects were prematurely withdrawn from the study due to adverse events. Additionally, 2 subjects were prematurely withdrawn from the study due to other reasons not related to the study.

Each active tablet contained the compound of formula (I) (1 mg or 2.5 mg), povidone (K30), light anhydrous silicic acid, carmellose calcium, crospovidone, and magnesium stearate. The placebo tablets were prepared in a similar manner except that the tablets did not contain the compound of formula (I).

Pharmacokinetic

Following a single the compound of formula (I) dose administration of 1 to 5 mg, plasma concentrations of the compound of formula (I) were generally below the limit of quantization on Day 1. On Day 14, the compound of formula (I) concentrations were not quantifiable at the 1 mg dose, whereas quantifiable concentrations were observed up to 5 hr for the 2.5 mg group (2 of 8 subjects) and the 3.5 mg dose group (7 of 8 subjects), and for at least 6 hr in the 5 mg dose group (2 of 3 subjects). For the dose groups that had quantifiable concentrations, the concentrations were generally close to the lower limit of quantization of the assay, and reached a maximum at 3 to 5 hr post-dose. The half-life of the compound of formula (I) could not be estimated due to lack of sufficient quantifiable concentrations in the parent drug concentration versus time profile. The compound of formula (I) appeared to be rapidly metabolized, such that, estimation of lambda-z (and therefore the half-life) was not possible. Cₘₐₓ and AUC_{0-τ}increased in an approximately dose proportional manner from 2.5 to 5 mg. Dose proportionality could not be satisfactorily concluded from a statistical perspective due to the wide range of confidence intervals for the slope of the log-linear regression analysis of Cₘₐₓ and AUC_{0-τ}(due to the large variability resulting from the low concentrations). The compound of formula (I) had a high oral clearance that was similar across the quantifiable dose groups. Overall, these data demonstrated that, following twice daily dosing for 14 days, systemic exposure to the compound of formula (I) was negligible. It is noted that in a previous human study (Example 2) with the compound of formula (I) administered at 5 mg BID for 14 days, substantial safety margins (80 to about 200-fold based on AUCs) for the compound of formula (I) were observed relative to exposure in a 3-month safety study in dogs at a dose of 25 mg/kg at which there were no toxicity findings.

M1 concentrations increased with ascending doses, and showed quantifiable concentrations for most of the observations on Day 1 and for the entire sampling period on Day 14. M1 exposure parameters [Cₘₐₓ and AUC_{0-τ}] exhibited a dose proportional increase from 1 to 5 mg. The Tₘₐₓ varied from 4 to 6 hr across the dose groups and was similar to that of the compound of formula (I), thereby indicating that the metabolite was rapidly formed. The parent/metabolite ratio of less than 0.01 for AUC_{0-τ3} and less than 0.03 for Cₘₐₓ indicates that metabolite M1 is the main circulating species and that systemic exposure of the compound of formula (I) is negligible at all dose levels. The half-life (t_{1/2}) estimates were similar for the 2.5 and 5 mg groups (32.9 to 41.1 hr) and appeared to be independent of dose. The mean t_{1/2} for the 1 mg group was 70.1 hr and ranged from 30.2 to 120.8 hrs, with most of the half-life estimates being higher than 50 hr. The longer half-life in the 1 mg dose group relative to the other dose groups is likely due to 4 subjects with M1 plasma concentrations close to the lower limit of quantization of the assay (0.300 ng/mL), resulting in an artificially long half-life (ranging from 85 to 120 hr). Accumulation of the M1 metabolite was extensive and ranged between 11.5 to 21.9 fold (based on AUC_{0-τ}) over the 14-day treatment period. The time to reach steady-state ranged from 7 days (1 and 2.5 mg groups) to 5 days (3.5 and 5 mg groups). The fraction excreted as metabolite ranged from 26.0 to 37.3% and was similar across dose groups. Mean renal clearance (CLr) in the 5 mg dose group was lower than other treatment groups, but the difference was not significant in view of the large inter-subject variability and smaller sample size.

Serum Lipid Levels

Postprandial levels of lipid parameters after administration of the compound of formula (I) were evaluated using the change from preceding timepoint. Following breakfast and dinner, postprandial estimates for TG, VLDL-TG, and chylomicron-TG post the compound of formula (I) dosing were substantially lower when compared with placebo. The postprandial decrease was greater on Days 10 and 14 compared with Day 1, suggesting that greater postprandial suppression is achieved following repeated administration of the compound of formula (I). Intraday changes and changes from pretreatment baseline also indicate that the absorption of TG, VLDL-TG, and chylomicron-TG were delayed compared to placebo. This delayed absorption is consistent with a previous pharmacology study in healthy male Japanese subjects. The postprandial results were interpreted with caution as the delay in absorption can impact the calculation regarding the magnitude of the postprandial change in triglycerides. However, the suppression of the expected increases in postprandial TG, VLDL-TG, and chylomicron-TG, which is indicative of MTP inhibition, clearly supports the mechanism of action of the compound of formula (I). The VLDL-TG changes observed in this study are consistent with previous studies (Examples 2 and 6) where the compound of formula (I) was administered for up to 14 days in healthy or hypertriglyceridemic Caucasian and Japanese male subjects. In general, the compound of formula (I) levels were also low or non-quantifiable in these studies. In addition, a previous study (Example 6) has shown that the compound of formula (I) affects campesterol (a marker of cholesterol absorption), but not lathosterol (a marker of cholesterol synthesis by the liver) without the presence of fatty liver (evaluated by liver MRI). Therefore, the compound of formula (I) is not believed to directly affect the liver.

Weight Loss

The inhibition of MTP by the compound of formula (I) resulted in a significant reduction in pretreatment baseline adjusted values of TC, LDL-C, LDL-TG, ApoB, and phospholipids. The decrease in LDL-C levels was accompanied by a corresponding decrease in ApoB levels, the predominant apolipoprotein associated with LDL-C. The decrease in levels appeared to be similar between Day 7 and Day 14, indicating that dosing up to 7 days may be sufficient to produce maximum inhibition of lipid levels. The decrease in HDL-C levels was not as pronounced as LDL-C, nor was it statistically significant. Therefore, the reduction of plasma TC levels compared with placebo appears to be primarily due to a decrease in cholesterol carried by LDL (LDL-C) and a small contribution of slightly reduced HDL-C levels.

The compound of formula (I) administration for 14 days also resulted in an average weight loss of 0.31 kg, 0.68 kg, 1.39 kg and 2.07 kg, respectively, for the 1 mg, 2.5 mg, 3.5 mg, and 5 mg dose groups. Although six (of nine) subjects withdrew from the study from the 5 mg dose group due to GI-related adverse events prior to Day 14, the 5 mg dose group had an average weight loss of 2.32 kg (n=9) at Day 10. The weight loss observed in this study is likely due to the suppression and delayed absorption of triglycerides, the increased incidence of GI-related adverse events resulting in changes in food intake behavior, or a combination of all of these factors.

Overall, the results of this study showed that in obese subjects, the compound of formula (I) was well tolerated at doses up to 3.5 mg BID, but not at 5 mg BID, and that these doses were safe; negligible systemic exposure to the compound of formula (I), due to rapid and extensive hydrolysis, supports its systemic safety. This effect, in conjunction with its mechanism-based suppression of postprandial TG absorption and related weight loss, evidences the usefulness of the compound of formula (I) for the treatment of obesity.

### Example 8

This example involved a clinical study of the compound of formula (I), which was a randomized, double-blind, placebo-controlled, parallel group study to evaluate the efficacy of the compound of formula (I) administered twice daily for twelve weeks in obese subjects.

The study had 180 participants. The 180 subjects comprised 59 males and 121 females, 18 to 65 years old, with BMI≧30 kg/m² to <40 kg/m². The subjects were separated into four arms of 45 subjects, including one arm as placebo. Each test subject received a tablet taken orally over a period of 12 weeks after breakfast and dinner (BID) (specifically, within 180 minutes of the commencement of eating) of 1 mg, 2.5 mg, or 3.5 mg, using one or multiples of tablets of 1 mg and 2.5 mg of the compound of formula (I) to achieve the total dose. Of the total entrants, 40 entrants completed the 1 mg regimen, 42 entrants completed the 2.5 mg regimen, 41 entrants completed the 3.5 mg regimen, and 42 entrants completed the placebo regimen.

One hundred sixty-five subjects (91.7% [42 subjects, 93.3% placebo; 40 subjects, 88.9% the compound of formula (I) 1 mg BID; 42 subjects, 93.3% the compound of formula (I) 2.5 mg BID; 41 subjects, 91.1% the compound of formula (I) 3.5 mg BID]) completed the 12-week treatment period. Fifteen subjects (3 placebo subjects, 5 the compound of formula (I) 1 mg BID subjects, 3 the compound of formula (I) 2.5 mg BID subjects, 4 the compound of formula (I) 3.5 mg BID subjects) did not complete the 12-week treatment period. There were no apparent differences in the reasons and percentages of subjects prematurely discontinuing across treatment groups.

The most common reason for premature discontinuation was withdrawal of voluntary consent (2 placebo subjects, 2 the compound of formula (I) 1 mg BID subjects, 1 the compound of formula (I) 3.5 mg BID subject), followed by biological adverse event (2 subjects the compound of formula (I) 2.5 mg BID, 2 subjects the compound of formula (I) 3.5 mg BID) and lost to follow-up (1 placebo subject, 3 the compound of formula (I) 1 mg BID subjects).

Each active tablet contained the compound of formula (I) (1 mg or 2.5 mg), povidone (K30), light anhydrous silicic acid, carmellose calcium, crospovidone, and magnesium stearate. The placebo tablets were prepared in a similar manner except that the tablets did not contain the compound of formula (I).

Weight Loss

This study was designed to investigate the weight loss effect of the compound of formula (I) administered BID for 12 weeks to obese subjects. The effect of the compound of formula (I) on ancillary efficacy measures (e.g., waist circumference, waist-hip ratio, percent body fat, serum lipid parameters, and markers related to glycemic control, obesity, and inflammation) was also evaluated, as was safety. The primary efficacy variable was body weight change from baseline after 12 weeks of treatment. Dose-dependent mean decreases from baseline to EOT in body weight were observed in the compound of formula (I) 1 mg BID, 2.5 mg BID, and 3.5 mg BID treatment groups (−0.70 kg, −1.74 kg, and −2.19 kg, respectively) compared to a mean increase from baseline in the placebo group (0.34 kg). The difference between the compound of formula (I) 2.5 mg BID and 3.5 mg BID treatment groups and the placebo group was statistically significant, and weight loss was maintained throughout the treatment period. These data compare favorably with a 2-week, multiple-dose study in obese subjects (Example 7), which reported mean changes from baseline in weight of −0.31 kg, −0.68 kg, and −1.39 kg in the compound of formula (I) 1 mg BID, 2.5 mg BID, and 3.5 mg BID treatment groups, respectively. Together, these data show that the compound of formula (I) treatment causes dose-dependent weight loss and that a longer treatment duration may lead to a more pronounced decrease in body weight. Weight loss was also examined based on perilipin genotype, as those with perilipin 11482G>A mutations have been shown to have decreased perilipin content, increased lipolytic activity, and greater resistance to weight loss in response to low-energy diets. However, in this study no differences were noted in weight loss between subjects with a mutation in their perilipin gene and those without such a mutation.

The efficacy of the compound of formula (I) compared with placebo was supported by results of secondary efficacy variables: percent change from baseline in body weight, and achievement of ≧2.5% reduction (the compound of formula (I) 2.5 mg BID and 3.5 mg BID) or ≧5.0% reduction in body weight (the compound of formula (I) 3.5 mg BID). Mean percent reduction in LDL-C was observed in all 3 the compound of formula (I) treatment groups. After 12 weeks of the compound of formula (I) administration, mean decreases from baseline LDL-C ranged from −5.90 to −8.27 mg/dL, while a mean increase of 2.63 mg/dL was noted in the placebo group. The magnitude of LDL-C reduction was comparable across all active dose groups, and was similar to that of a low-fat diet.

Serum Lipid Levels

In addition to LDL-C, a protocol-specified secondary efficacy variable, other lipid parameter levels were measured as additional efficacy endpoints. Triglyceride levels increased from baseline in all 4 treatment groups, although high variability was observed. Similar to a 2-week, multiple-dose study with the compound of formula (I), improvements (reductions) in TC and ApoB, the predominant apolipoprotein associated with LDL-C, were observed. However, due to the large variability in lipid parameter results in this study, no meaningful conclusions could be made for lipid parameter results, with the exception of LDL-C, as described above.

No statistically significant differences between any the compound of formula (I) treatment group and placebo were observed after 12 weeks of treatment for mean change or mean percent change from baseline in anthropometric parameters (waist circumference and waist-hip ratio), or percent body fat.

Pharmacokinetic

The compound of formula (I) trough concentrations were not quantifiable in the 1 mg BID dose group. In the 2.5 and 3.5 mg BID groups, the compound of formula (I) was quantifiable in a few subjects, at levels close to the lower limit of quantification of the assay. These observations were expected, as they demonstrate the low/negligible exposure to the compound of formula (I) due to its rapid hydrolysis. Mean trough concentration of the main inactive metabolite, M1, increased in a dose-related manner and, within each treatment group, there was no trend in treatment-duration related changes during the 12-week treatment period.

Safety

The compound of formula (I) was generally well tolerated across the dose range evaluated in this study. The most frequently reported treatment-emergent adverse events were gastrointestinal in nature, with diarrhea being the most common adverse event in each treatment group. The incidence of diarrhea was generally highest during the first month of treatment and decreased over time. The majority of the events of diarrhea in each treatment group were described as loose stool. In addition, although the majority of subjects experienced diarrhea, only 3 subjects (1 the compound of formula (I) 2.5 mg BID subject, 2 the compound of formula (I) 3.5 mg BID subject) prematurely discontinued due to diarrhea.

Another important treatment-emergent adverse event was decreased appetite. The incidence of decreased appetite that was considered to be drug-related appeared to increase dose-dependently: 2.2% (1/45 subjects) in the placebo group, 4.4% (2/45 subjects) in the compound of formula (I) 1 mg BID group, 20.0% (9/45 subjects) in the compound of formula (I) 2.5 mg BID group, and 22.2% (10/45 subjects) in the compound of formula (I) 3.5 mg BID group. These adverse events were expected based on the mechanism of action of the compound of formula (I).

Previous MTP inhibitors have been monitored for fatty liver and vitamin malabsorption. With the compound of formula (I), although mean increases in liver function parameters (AST, ALT, and alkaline phosphatase) were detected in some the compound of formula (I) treatment groups, no subject had a PCS value. There were also no clinically significant changes in the fat-soluble vitamins, or coagulation factors (which may be dependent on Vitamin K). These observations support the hypothesis that the compound of formula (I) inhibits intestinal MTP without affecting the liver or the absorption of fat-soluble vitamins.

### Example 9

This example involved a clinical study of the compound of formula (I), which was a randomized sequence, double-blind, placebo-controlled, multiple dose, cross-over study evaluating the safety, tolerability, and pharmacodynamics of the compound of formula (I) administered for 10 days in obese male subjects.

The effect of the compound of formula (I) on gut hormone levels, appetite, and caloric/food intake was studied. The study enrolled 16 obese male subjects (18-65 years of age (inclusive), with a body mass index (BMI) >30.0 kg/m² and <40.0 kg/m²) who participated in two treatment periods. During Period 1, 8 subjects were randomized to receive 3.5 mg of the compound of formula (I), and 8 subjects were randomized to receive placebo. The compound of formula (I) or placebo was administered twice daily (BID), 30 minutes after the start of breakfast and dinner, for 10 days. All subjects were discharged from the clinic at the end of Period 1 for a 9±2 day discharge period. Subjects then returned for treatment during Period 2, in which (i) the group previously receiving the compound of formula (I) now received placebo, and (ii) the group previously receiving placebo now received the compound of formula (I), in accordance with the protocol described above.

Each active tablet contained either 1 mg or 2.5 mg of the compound of formula (I) and the following inactive ingredients: povidone (K30), light anhydrous silicic acid, carmellose calcium, crospovidone, and magnesium stearate. Each placebo tablet contained the following ingredients: povidone (K30), light anhydrous silicic acid, carmellose calcium, crospovidone, and magnesium stearate.

Gut Hormone Levels (Pharmacodynamics)

The primary objective of this study was to examine the change in gut hormone levels. The co-primary endpoints were trough gut hormone (glucagon-like peptide 1 (GLP-1) active, peptide tyrosine tyrosine (PYY) total, and gastric inhibitory polypeptide (GIP) total) levels on Day 10 in each treatment period and the trough gut hormone profile throughout the study. On Day 10 of each treatment period, statistically significant mean increases in GLP-1 active (60%) and PYY total (43%) were observed during administration of the compound of formula (I) compared with placebo, whereas no change in GIP total relative to placebo was found. Similarly, trough gut hormone levels of GLP-1 active and PYY total showed statistically significant increases at the majority of timepoints throughout the study after administration of the compound of formula (I) compared with placebo (GLP-1 active: average increase 45% from days 3 through 11, PYY total: average increase 63% from days 2 through 11), while statistically significant increases in trough GIP total were only detected on days 3, 5 and 11 with an average increase of 24%.

The effectiveness of the compound of formula (I) compared with placebo in inducing gut hormones was further supported by one of the secondary endpoints, which assessed levels 4 hours after dinner (on Days 2 and 9 of each period). Administration of the compound of formula (I) resulted in significant increases at 4 hours after dinner on Day 2 and Day 9 of each period in GLP-1 active (38% and 71%, respectively) and PYY total (44% and 42%, respectively) compared with placebo. No statistically significant differences in GIP total were observed relative to placebo at 4 hours post-dinner on either day.

Exploratory pharmacodynamic endpoints included gut hormone profiles on Days 2 and 9 of each period around breakfast (between −0.5 hours and 3.5 hours relative to dosing), gut hormone profile differences between Day 2 and Day 9 of dosing, and gut hormone AUC profile between −0.5 and 3.5 hours relative to dosing. While no significant changes in AUC profiles around breakfast (between −0.5 hours and 3.5 hours relative to dosing) were noted on Day 2 for any of the gut hormones, the AUC for GLP-1 active and PYY total on Day 9 significantly increased (31% and 39%, respectively) after administration of the compound of formula (I) compared with placebo, and these increases were maintained up to 4 hours post-breakfast. For GIP total, the AUC around breakfast on Day 9 showed a small, but significant, decrease (17%) compared with placebo. On Day 2, administration of the compound of formula (I) did not significantly affect any of the gut hormone levels. On Day 9, GLP-1 active and PYY total levels around breakfast were consistently higher following administration of the compound of formula (I) compared with placebo, with significant differences observed at the majority of timepoints. The average increase around breakfast on Day 9 was 38% for GLP-1 active and 51% for PYY total. No significant changes in the GIP total profile following administration of the compound of formula (I) were noted on Day 2 or Day 9.

Caloric/Food Intake

A secondary objective of this study was to examine the effects on caloric/food intake and appetite. Therefore, other secondary PD endpoints in this study included caloric intake and meal weight consumed on Day 10 of each period, as well as visual analog scale (VAS) assessments of hunger, prospective consumption, and fullness on Day 10/11 of each period. VAS assessments of thirst and nausea on Day 10/11 of each period were part of the exploratory analysis. However, no statistically significant treatment differences were observed for these parameters.

Safety and Tolerability

The percentage of subjects experiencing at least one treatment-emergent adverse event was similar in the placebo and the compound of formula (I) 3.5 mg BID treatment groups (50.0% and 53.3%, respectively). The most commonly reported treatment-emergent adverse events were associated with the gastrointestinal and nervous systems. The most common (>1 subject in either treatment group) gastrointestinal disorders included diarrhea, nausea, vomiting, and abdominal pain. The most common (>1 subject in either treatment group) nervous system disorders included dizziness and headache. All treatment-emergent adverse events were considered to be mild or moderate in severity, and the majority of treatment-emergent adverse events were considered not to be related to the compound of formula (I).

There were no deaths or serious adverse events during the study. One subject prematurely discontinued the study due to treatment-emergent adverse events of dizziness, abdominal pain, diarrhea, and vomiting. No clinically meaningful mean changes from baseline were observed with respect to hematology, biochemistry, urinalysis, vital sign (including weight), and electrocardiogram (ECG) results.

A 10-day administration of the compound of formula (I) 3.5 mg BID under fed conditions was safe and well-tolerated in obese, otherwise healthy, male subjects.

### Example 10

This example sets forth a clinical study of the compound of formula (I), which is a randomized, double-blind, placebo-controlled, multi-center, parallel group study evaluating the efficacy, safety, and tolerability of the compound of formula (I) administered twice daily for 24 weeks in treatment-naive, metformin only or metformin plus sulfonylurea-treated obese type 2 diabetic patients.

This study will assess the effect of the compound of formula (I) on HbAlc and on other parameters associated with metabolic syndrome (e.g., fasting plasma glucose, plasma insulin, serum lipid parameters, weight loss, and waist circumference). Upon completion of the screening period, patients will enter a two-week single-blind placebo run-in period where they will be advised to follow a locally-accepted diet and exercise program for type 2 diabetics throughout the study. At the completion of the single-blind placebo run-in period, eligible patients will be randomized into one of four parallel dose groups (the compound of formula (I) 1 mg BID, the compound of formula (I) 3.5 mg BID, the compound of formula (I) 5 mg BID, and matching placebo BID) and will receive treatment for 24 weeks. Randomization will be stratified by region (US or rest of world [ROW]), baseline antidiabetic therapy (treatment naïve, metformin only, or metformin plus sulfonylurea) and HbAlc level at Visit 1 (HbAlc<8.5% versus HbAlc≧8.5%). This is a fixed-dose study, where patients will receive the same dose regimen from randomization to end of treatment with one exception: during the first two weeks of the double-blind treatment period, the patients randomized into the compound of formula (I) 5 mg BID dose group will be administered the compound of formula (I) 2.5 mg BID, followed by up-titration to the full 5 mg BID dose in Week 3. Patients with persistent hyperglycemia may be eligible for rescue therapy.

A sufficient number of patients will be screened to ensure the randomization (1:1:1:1) of approximately 460 patients (115 in each treatment group) and will include the following characteristics:

Males and females, 18-70 years of age (inclusive) at Visit 1 with a stable weight (i.e., no known weight variation of >5% within 3 months prior to Visit 1);

Body mass index (BMI) ≧27.0 kg/m² and ≦45.0 kg/m² at Visit 1;

Have type 2 diabetes (HbAlc between 7.0% and 10.0% [inclusive]) at Visit 1;

Have fasting plasma glucose levels ≦280 mg/dL (15.5 mmol/L) at Visit 1;

Are either drug naïve with respect to hypoglycemic agents (e.g., managed with diet and exercise only) or are currently being treated with metformin (at least 1500 mg/day) alone or in combination with a sulfonylurea (at least half the maximum labeled daily dose or at the maximum tolerated dose). Metformin and sulfonylurea dosing must be stable, defined as no change in dose for at least 2 months prior to Visit 1 and no expected change during the study;

Have serum triglyceride levels ≦500 mg/dL (5.6 mmol/L) at Visit 1;

Weight loss or ≦2.0% body weight gain between Visits 2 and 3;

Demonstrate good study drug compliance (80% to 120%) during the single-blind two-week placebo run-in period.

Each active tablet will contain either 1 mg or 2.5 mg of the compound of formula (I) and the following inactive ingredients: povidone (K30), light anhydrous silicic acid, carmellose calcium, crospovidone, and magnesium stearate. Each placebo tablet will contain the following ingredients: povidone (K30), light anhydrous silicic acid, carmellose calcium, crospovidone, and magnesium stearate.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating or preventing a disorder in a subject comprising administering twice per day a therapeutically effective amount of a compound of formula (I) 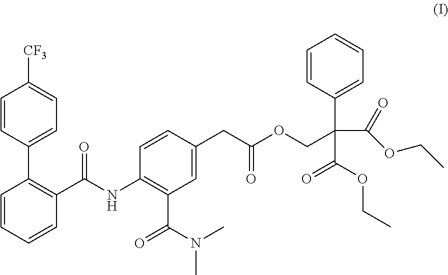
or a pharmaceutically acceptable salt, solvate, or hydrate thereof, to a subject that has or is at risk of having a disorder selected from the group consisting of hyperlipidemia, arteriosclerosis, coronary artery diseases, obesity, diabetes and hypertension, whereupon the disorder is treated or prevented in the subject.

2-11. (canceled)

12. The method of claim 1, wherein the method comprises administering twice per day a therapeutically effective amount of the compound of formula (I), or a pharmaceutically acceptable salt, solvate, or hydrate thereof, to the subject, and the therapeutically effective amount is increased once during the course of the administration of the compound of formula (I), or a pharmaceutically acceptable salt, solvate, or hydrate thereof.

13. The method of claim 12, wherein the method comprises
(a) administering twice per day a first therapeutically effective amount of the compound of formula (I), or a pharmaceutically acceptable salt, solvate, or hydrate thereof, to the subject for a first time period, and
(b) administering twice per day a second therapeutically effective amount of the compound of formula (I), or a pharmaceutically acceptable salt, solvate, or hydrate thereof, to the subject for a second time period after the first time period,
wherein the second therapeutically effective amount is greater than the first therapeutically effective amount.

14. The method of claim 13, wherein the first time period is about 1 to 5 weeks.

15-26. (canceled)

27. The method of claim 13 or 14, wherein the second therapeutically effective amount is at least 25% greater than the first therapeutically effective amount.

28. The method of claim 13 or 14, wherein the second therapeutically effective amount is at least 50% greater than the first therapeutically effective amount.

29. The method of claim 13 or 14, wherein the second therapeutically effective amount is at least 75% greater than the first therapeutically effective amount.

30. The method of claim 13 or 14, wherein the second therapeutically effective amount is at least 100% greater than the first therapeutically effective amount.

31. The method of claim 1, wherein the method comprises administering twice per day a therapeutically effective amount of the compound of formula (I), or a pharmaceutically acceptable salt, solvate, or hydrate thereof, to the subject, and the therapeutically effective amount is increased twice during the course of the administration of the compound of formula (I), or a pharmaceutically acceptable salt, solvate, or hydrate thereof.

32. The method of claim 31, wherein the method comprises
(a) administering twice per day a first therapeutically effective amount of the compound of formula (I), or a pharmaceutically acceptable salt, solvate, or hydrate thereof, to the subject for a first time period,
(b) administering twice per day a second therapeutically effective amount of the compound of formula (I), or a pharmaceutically acceptable salt, solvate, or hydrate thereof, to the subject for a second time period after the first time period, and
(c) administering twice per day a third therapeutically effective amount of the compound of formula (I), or a pharmaceutically acceptable salt, solvate, or hydrate thereof, to the subject for a third time period after the second time period,
wherein
the second therapeutically effective amount is greater than the first therapeutically effective amount, and
the third therapeutically effective amount is greater than the second therapeutically effective amount.

33. The method of any one of claims 32, and 46-53, wherein the first and second time periods are each independently about 1 to 5 weeks.

34-45. (canceled)

46. The method of claim 32, wherein the second therapeutically effective amount is at least 25% greater than the first therapeutically effective amount.

47. The method of claim 32, wherein the second therapeutically effective amount is at least 50% greater than the first therapeutically effective amount.

48. The method of claim 32, wherein the second therapeutically effective amount is at least 75% greater than the first therapeutically effective amount.

49. The method of claim 32, wherein the second therapeutically effective amount is at least 100% greater than the first therapeutically effective amount.

50. The method of claim 32, wherein the third therapeutically effective amount is at least 25% greater than the second therapeutically effective amount.

51. The method of claim 32, wherein the third therapeutically effective amount is at least 50% greater than the second therapeutically effective amount.

52. The method of claim 32, wherein the third therapeutically effective amount is at least 75% greater than the second therapeutically effective amount.

53. The method of claim 32, wherein the third therapeutically effective amount is at least 100% greater than the second therapeutically effective amount.

54-56. (canceled)

57. The method of any one of claims 1, 12-14, 31, 32 and 46-53, wherein the compound is administered to the subject when the subject is in the fed state.

58-66. (canceled)
